(19) United States
(12) Patent Application Publication
(10) Pub. No.: US 2023/0321101 A1
(43) Pub. Date: Oct. 12, 2023

(54) METHODS AND USES OF HALOFUGINONE

(71) Applicants: Philip L.S.M. GORDTS, San Diego, CA (US); Andrea DENARDO, GardoneValTrompia, CA (US); Ryan Joseph WEISS, Athens, CA (US); Chelsea NORA, San Diego, CA (US); Jeffrey D. Esko, San Diego, CA (US); CLAUSEN Thomas Mandel, San Diego, CA (US); Daniel R. SANDOVAL, San Diego, CA (US); The Regents of the University of California, Oakland, CA (US)

(72) Inventors: Philip L.S.M. Gordts, San Diego, CA (US); Andrea Denardo, Gardone Val Trompia (IT); Ryan Joseph Weiss, Athens, GA (US); Chelsea Nora, San Diego, CA (US); Jeffrey D. Esko, San Diego, CA (US); Thomas Mandel-Clausen, San Diego, CA (US); Daniel R. Sandoval, San Diego, CA (US)

(21) Appl. No.: 18/022,254

(22) PCT Filed: Aug. 20, 2021
(86) PCT No.: PCT/US2021/046965

§ 371 Date: Feb. 20, 2023

Related U.S. Application Data
(60) Provisional application No. 63/138,151, filed on Jan. 15, 2021.
(60) Provisional application No. 62/706,512, filed on Aug. 21, 2020.
(60) Provisional application No. 63/089,976, filed on Oct. 9, 2020.

(51) Int. Cl. A61K 31/517; A61P 31/14

(52) U.S. Cl. CPC A61K 31/517; A61P 31/14

(57) ABSTRACT

The present disclosure relates to treatment or prevention of a disease, such as COVID-19, in a subject by administering to the subject a therapeutically effective amount of halofuginone or a derivative or pharmaceutically acceptable salt thereof.

### CLAIM OF PRIORITY

This application claims the benefit of priority to U.S. Patent Application Ser. No. 62/706,512, filed on Aug. 21, 2020, and to U.S. Patent. Application Ser. No. 63/089,976, filed on Oct. 9, 2020, and to U.S. Patent Application Ser. No. 63/138,151, filed on Jan. 15, 2021, each of which is incorporated by reference herein in its entirety.

### BACKGROUND

The severe acute respiratory syndrome-related coronavirus 2 (SARS-CoV-2), the causative agent of COVID-19, is rapidly sweeping across the globe, causing morbidity and death and widespread disruption to all aspects of society. SARS-CoV-2 has caused 32.1 million infections and 980,339 confirmed deaths as of Sep. 2, 2020⁵. In May 2020, the US Food and Drug Administration (FDA) granted an Emergency Use Authorization (EUA) for Remdesivir (RDV, GS-5734) for the treatment of hospitalized patients with severe COVID-19⁶. In the preliminary report of the ACTT-1 clinical trial subgroup analysis, Remdesivir significantly reduced time to recovery only in patients who were on low-flow oxygen at baseline⁴. This finding suggested that Remdesivir treatment in COVID-19 may provide greater benefit if started before the development of severe disease^{7,8}. However, data from multiple trials suggests that Remdesivir provides modest clinical benefit compared with standard of care^{4,9,10}. Furthermore, Remdesivir must be administered intravenously, which functionally prevents its use in pre-symptomatic or early symptomatic mild disease. Convalescent plasma from individuals who have recovered from COVID-19 has also been granted EUA for hospitalized patients with COVID-19 but efficacy data from randomized trials are needed.

Currently, there is an urgent unmet clinical need for therapeutics that prevent and inhibit viral infection and dissemination. More specifically, there is an urgent unmet clinical need for antiviral therapeutics that can reduce COVID-19 associated morbidity and mortality and that optimally could be administered early after symptom onset to prevent the development of severe respiratory disease^{11,12}.

Many viral pathogens utilize glycans on the glycocalyx as attachment factors to facilitate the initial interaction with host cells, including influenza virus, Herpes simplex virus, human immunodeficiency virus, and different coronaviruses (SARS-CoV-1 and MERS). Cells are covered in a dense mesh of glycans termed the glycocalyx. Given the abundance and accessibility of the glycocalyx at the cell surface, it is not surprising that the glycocalyx acts as the first cellular contact point for a myriad of growth factors, cell surface receptors, and viruses. SARS-CoV-2 entry into lung upper airway epithelial cells depends on ACE2, TMPRSS2, and cell surface heparan sulfate (HS)^{3,13,14}. Blocking the interaction of cell surface HS and spike protein attenuate SARS-CoV-2 binding and SARS-CoV-2 infection³. These findings identify cellular HS as a necessary co-factor for SARS-CoV-2 infection and emphasize the potential for targeting S protein-HS interactions to attenuate virus infection.

The generation and discovery of small molecules that manipulate cellular heparan sulfate biosynthesis provide a unique clinical challenge.

### SUMMARY

The present disclosure provides in various embodiments a method of treating a subject suffering from a disease or condition selected from the group consisting of a virus, a coronavirus, an iron-loading disease, an iron-deficiency disease, a lysosomal storage disease, a neurodegenerative disorder, a cancer, diabetes, or need for wound healing, comprising administering to the subject a therapeutically effective amount of halofuginone or a pharmaceutically acceptable salt thereof.

The present disclosure also provides in embodiments a method of treating COVID-19 in a subject in need thereof. The method comprises administering to the subject a therapeutically effective amount of halofuginone or a pharmaceutically acceptable salt thereof.

In additional embodiments, present disclosure provides a method of treating COVID-19 in a subject in need thereof comprising administering to the subject a therapeutically effective amount of 2R,3S-(+) halofuginone or a pharmaceutically acceptable salt thereof.

In still further embodiments, the present disclosure provides a method of treating COVID-19 in a subject in need thereof comprising administering to the subject a therapeutically effective amount of 2R,3S-(+) halofuginone or a pharmaceutically acceptable salt thereof, or a pharmaceutical composition comprising a therapeutically effective amount of 2R,3S-(+) halofuginone or a pharmaceutically acceptable salt thereof.

The present disclosure also relates to halofuginone (HF) and Prolyl-tRNA Synthetase (PRS) inhibitors as potent inhibitors of SARS-CoV-2 attachment, infection, and replication. SARS-CoV-2 spike protein interacts with cell surface heparan sulfate and angiotensin-converting enzyme 2 (ACE2) through its Receptor Binding Domain (FIG. 36). In vitro studies confirm that HF and PRS inhibitors prevent cell surface presentation and production of heparan sulfate. Polysome sequencing, RNA sequencing and bioinformatic analyses confirm that inhibition occurs via blocking protein translation of essential heparan sulfate biosynthetic genes and core proteins, including syndecan and glypicans, as they are proline-rich. The activation of the integrated stress response and subsequent activation of ATF4 as a result of PRS inhibition was not responsible for the inhibition of heparan sulfate presentation and heparan sulfate-mediated SARS-CoV-2 infection. However, HF and PRS inhibitors-mediated activation of the integrated stress response and the resulting inhibition of 5′cap-RNA translation was responsible for ultimately preventing replication and propagation of SARS-CoV-2 in human lung airway epithelial cells. These findings support a model wherein manipulation of PRS via HF and other compounds, as well as targeting activation of the integrated stress response, can be leveraged as antiviral medications to prevent SARS-CoV-2 attachment, infection, and replication.

### BRIEF DESCRIPTION OF THE DRAWINGS

FIG. 1A-FIG. 1J. Screen of epigenetic and translational regulatory compounds identify Halofuginone as a potent inhibitor of SARS-CoV-2 Spike HS dependent cellular adhesion. A, Hep3B cells were treated with a library of epigenetic and translational regulatory compounds or heparin lyase (HSase) as a positive control and tested for their interaction with recombinant SARS-CoV-2 RBD protein. B-E, Titration of halofuginone on Hep3B cells (B), Vero E6 (C), Calu-3 (D) and Cacu-2 (E) cells (n=2-4 replicates per condition), and its effect on binding of recombinant SARS-CoV-2 RBD protein Heparin Lyase treatment is included as control for HS dependent adhesion. F-G, effect of halofuginone treatment on the infection of SARS-CoV-2 spike protein pseudotyped virus in Hep3B (F) and Vero E6 (G) cells (n=3-4 replicates per condition). H, Authentic SARS-CoV-2 virus infection of Hep3B cells treated with halofuginone. I, Infection of primary human bronchial epithelial cells, grown at an air-liquid interface, treated with halofuginone. J, Relative cell viability of primary human bronchial epithelial cells. Data shown as mean±S.D. Statistics performed by 1-way ANOVA and uncorrected Fisher's LSD test (ns: p>0.05, *:p≤0.05, **:p≤0.01, ***:p≤0.001).

FIG. 2A-FIG. 2G. Halofuginone Inhibits Heparan Sulfate Biosynthesis. A, Schematic representation of HS biosynthesis. Genes required for priming and elongation are highlighted in black. Sulfotransferases and other modifying enzymes are highlighted in red. B, Schematic example of the interaction with the anti-HS 10E4 and 3G10 mAb's. 10E4 recognizes sulfated HS polymer, while 3G10 recognizes the number of HS attachments sites by interacting with the stub left after heparin lyase (HSase) treatment. C-D, titration of halofuginone on Hep3B cells (C), Vero E6 (D), and its effect on cellular staining with anti-HS 10E4 mAb (n=3 per group). E, quantitative determination of HS content in Hep3B cells treated with halofuginone. Absolute HS content was determined by disaccharide analysis using LC-MS (n=3 per group). F, Qualitative distribution of specific sulfation patterns in Hep3B cells treated with halofuginone, seen in the LC-MS analysis (n=3 per group). G, Quantification of functional HS binding sites in Hep3B cells treated with halofuginone. Binding sites are quantified in SDS-PAGE using mAb 3G10 that recognizes the linker tetrasaccharide bound to the HSPG core protein after hep lyase treatment. Data shown as mean±S.D. Statistics performed by unpaired t test or 1-way ANOVA and uncorrected Fisher's LSD test (ns: p>0.05, *:p≤0.05, **:p≤0.01, ***:p≤0.001, ****:p≤0.0001).

FIG. 3A-FIG. 3I. Halofuginone Inhibition of heparan sulfate presentation and Spike protein binding is not dependent of the integrated stress response. A, Schematic representation of the mediators of the integrated stress response (ISR). B, Chemical structure of halofuginone and negative control compound MAZ1310. Figure shows the interaction of halofuginone with the human prolyl-tRNA synthetase (PRS) active site, as resolved by X-ray crystallography (PDB: 4K88)⁴⁸. C, Chemical structure of ProSA. Graphic shows the interaction of ProSA with human PRS (PDB: 5V58)⁴⁹. D-E, Treatment of Hep3B cells with modulators of the PRS pathway at 0.5 μM (ProSA at 5 μM) and its effect on (D) HS presentation as measured by anti-HS mAb 10E4 binding and (E) spike RBD binding by flow cytometry. Binding is represented as relative to non-treated control. F, Treatment of Hep3B cells with modulators of the halofuginone with or without 4 mM proline and its effect on spike RBD binding by flow cytometry. Binding is represented as relative to non-treated control. G, Treatment of Hep3B cells with modulators of the ISR and its effect on HS presentation as measured by anti-HS mAb 10E4 binding in flow cytometry. H, Treatment of Hep3B cells with modulators of the ISR and its effect on SARS-CoV-2 recombinant protein binding in flow cytometry. Binding is represented as relative to non-treated control. I, The distribution of proline distribution and density depicted as a proline distribution score for collagens, cholesterol biosynthetic proteins, lysosomal proteins, heparan sulfate (HS) biosynthetic proteins, heparan sulfate proteoglycans (HSPG) and viral host factor proteins (##p≤0.01 collagens vs. all other protein classes). Data shown as mean±S.D. Statistics performed by 1-way ANOVA and uncorrected Fisher's LSD test (ns: p>0.05, *:p≤0.05, **:p≤0.01, ***:p≤0.001, ****:p≤0.0001; #:p≤0.05, ##:p≤0.01).

FIG. 4A.-FIG. 4K. Halofuginone Inhibits infection and replication of authentic SARS-CoV-2. A-B, Authentic SARS-CoV-2 virus infection of Huh7.5 cells treated with Halofuginone. Huh7.5 cells treated with halofuginone pre- or post-infection, or both pre- and post-infection with authentic SARS-CoV-2 virus. Viral titers (A) and quantification of viral RNA (B) in the infected cells is shown. C, Immunofluorescent quantification of viral nucleocapsid (red) protein in Vero E6 cells treated with Halofuginone and Remdesivir and infected with authentic SARS-CoV-2 virus (nuclei=green). D, Authentic SARS-CoV-2 virus infection of Vero E6 cells treated with Halofuginone and Remdesivir measured in flow cytometry. E, Quantification of plaque formation and viral RNA in Vero E6 cells treated with Halofuginone and Remdesivir and infected with authentic SARS-CoV-2 virus. F, Rescue experiment of the effect of halofuginone treatment on SARS-CoV-2 infection using excess proline. G, Treatment of Vero E6 cells with halofuginone and enantiomers and their effect on SARS-CoV-2 infection. H, Treatment of Vero E6 cells with modulators of the PRS pathway and their effect on SARS-CoV-2 infection. I, Treatment of Vero E6 cells with modulators of the ISR pathway and their effect on SARS-CoV-2 infection. J, The distribution of proline distribution and density depicted as a proline distribution score for collagens, cholesterol biosynthetic proteins, lysosomal proteins and viral SARS-CoV-2 (SARS2), SARS-CoV-1 (SARS1), MERS-CoV (MERS), HCoV-229E and HCoV-LN63 proteins (##p≤0.01 collagens vs. all other protein classes). K, Treatment of Vero E6 cells with AARS inhibitors and their effect on SARS-CoV-2 infection. Data shown as mean±S.D. Statistics performed by 1-way ANOVA and uncorrected Fisher's LSD test (ns: p>0.05, *:p≤0.05, **:p≤0.01, ***:p≤0.001, ****:p≤0.0001, ##p:≥0.01.

FIG. 5A-FIG. 5G Halofuginone Inhibits Live SARS-CoV-2 infection and Replication.

FIG. 6. Hep3B—18 hr incubation. Halofuginone inhibits basal hepcidin expression in human hepatocytes

FIG. 7. Hep3B—24 hr incubation. Halofuginone inhibits BMP6 induced expression of hepcidin in human hepatocytes.

FIG. 8A and FIG. 8B. HepG2—24 hr incubation. Halofuginone inhibits BMP induced expression of hepcidin and increases SMAD7 levels, an inhibitor of hepcidin expression, in human hepatocytes.

FIG. 9. HepG2—24 hr incubation: Halofuginone inhibits inflammation (1L6) induced expression of hepcidin in human hepatocytes

FIG. 10. In vivo data: Halofuginone reduces hepcidin expression in the mouse liver upon iron loading with an iron-rich diet. Mice treated with Halofuginone for 8 days, 1 μg per mouse per day. Mice were either on normal iron-balanced chow (0.2 g/kg) or iron-rich diet (8.3 g/kg) for 7 days in conjunction with the treatment.

FIG. 11. COVID epigenetic screen.

FIG. 12. SARS CoV2 RBD binding.

FIG. 13. Geometric mean of FL-4.

FIG. 14. Spike protein.

FIG. 15. Halofuginone treats COVID-19 infection.

FIG. 16. Heparan promotes targeting.

FIG. 17. Halofuginone effect on 10E4 binding.

FIG. 18. Halofuginone effect on HAMP/GAPDH expression.

FIG. 19A-FIG. 19B. Compound Library and Working Concentrations. Epi, epigenetic inhibitor; Kin, Kinase inhibitor; PSR, Prolyl-tRNA synthetases inhibitor.

FIG. 20A-20C. SARS-CoV-2 infection and cell viability after SARS-CoV-2 infection and Halofuginone treatment in primary human airway lung epithelial cells. (A) Flow cytometry analysis for SARS-CoV-2 Viral nucleocapsid (conjugated to Alexa594) of primary human airway lung epithelial cells uninfected and infected with SARS-CoV-2 and treated with DMSO or 100 nM halofuginone. (B) Absolute percentage of infection with authentic SARS-CoV-2 of human bronchial epithelial cells, grown at an air-liquid interface, treated with Halofuginone as measured by flow cytometry. (C) Absolute cell viability of human bronchial epithelial cells, grown at an air-liquid interface, treated with Halofuginone and measured with authentic SARS-CoV-2 as measured by flow cytometry Represented are two independent experiments. Data shown as mean±S.D.

FIG. 21. Evaluation of 10E4 Binding to Hep3B wildtype and Hep3B NDST1-knockout cells. Titration of halofuginone on wild-type Hep3B and NDST1-deficient (NDST1−/−) cells and its effect on cellular staining with anti-HS 10E4 mAb. HSase, Heparin Lyases. Data are expressed as mean±S.D. Statistics performed by 1-way ANOVA (ns: p≥0.05, ***:p≤0.001).

FIG. 22. Western blot analysis for β-ACTIN. Loading control β-actin for SDS-PAGE using mAb 3G10 that recognizes the linker tetrasaccharide bound to the HSPG core protein after hep lyase treatment in FIG. 2G.

FIG. 23A-FIG. 23G. Halofuginone Inhibits Heparan Sulfate Proteoglycan Expression. A-B, RNA-Seq analysis and quantification of differentially expressed genes in Hep3B cells treated with Halofuginone at 200 nM and 500 nM for 6 and 18 h (n=2-3 per group). C, Gene annotation analysis of downregulated genes at 200 nM Halofuginone treatment for 6 h. D, Quantification of genes involved in the ATF4-mediated integrated stress response (ISR) versus genes affected by the ER stress response (ER-SR) upon treatment with Halofuginone (average of n=3 per group). E, Gene annotation analysis of upregulated genes at 200 nM Halofuginone treatment for 6 h. F, Quantification of a select group of HS biosynthetic enzymes and core HSPGs genes (average of n=3 per group). G, Time dependent effects of Halofuginone treatment on expression of HS related biosynthetic enzymes, HSPG core proteins, and sulfate transporters. PC: Principal Component (n=2-3 per group).

FIG. 24. Transcriptome analysis of HPSE and SULF1 expression. Quantification of a select group of HS processing enzymes previously shown to be affected by halofuginone in other cell types (l) (average of n=2-3 per condition).

FIG. 25A-FIG. 25C. Proline distribution in cholesterol biosynthetic enzymes, lysosomal proteins, and collagens. Proline location and kernel density, estimation of proline distribution within cholesterol biosynthetic enzymes (A), lysosomal proteins (B), and collagen proteins (C). Each black line indicates the location of a proline residue in the protein. The red curves show the kernel density estimation of proline distribution within a protein, which indicates the estimated probability that a proline residue will be found in each location within a protein. Kernel density estimates were calculated using the statistical software package R (see Examples).

FIG. 26A-FIG. 26B. Prolific Distribution of Heparan Sulfate Biosynthetic Genes and Viral Host Factors. Proline location and kernel density estimation of proline distribution within (A) heparan sulfate proteoglycans and (B) viral host factors, including beta-actin (ACTB). Each black line indicates the location of a proline residue in the protein. The red curves show the kernel density estimation of proline distribution within a protein, which indicates the estimated probability that a proline residue will be found in each location within a protein. Kernel density estimates were calculated using the statistical software package R (see Examples).

FIG. 27A-FIG. 27C. Proline distribution in heparan sulfate biosynthetic enzymes. Heparan Sulfate proteoglycans and heparan sulfate processing enzymes. Proline location and kernel density estimation of proline distribution within heparan sulfate biosynthetic enzymes (A), heparan sulfate proteoglycans (B), and processing enzymes (C). Each black line indicates the location of a proline residue in the protein. The red curves show the kernel density estimation of proline distribution within a protein, which indicates the estimated probability that a proline residue will be found in each location within a protein. Kernel density estimates were calculated using the statistical software package R (see Examples).

FIG. 28. Cell viability in Huh 7.5 cells after treatment for 24 hours with Halofuginone. Cell viability was measured using alamarBlue staining in HuH 7.5 cells at indicated concentrations of halofuginone after a 24 hr treatment (n=3 per dose). Data shown as mean±S.D. Statistics performed by 1-way ANOVA (ns: p>0.05, *:p≤0.01).

FIG. 29A and FIG. 29B. Cell viability after SARS-CoV-2 infection and treatment with Halofuginone and proline for 24 hours in Vero E6 cells. (A) Cell viability was measured using Lactate Dehydrogenase (LDH) in media in Vero E6 cells at indicated concentrations of halofuginone after a 24 hr treatment (n=3 per dose). (B) Cell number was measured using Lactate Dehydrogenase (LDH) after triton-X addition for 10 min at room temperature to fresh DMEM to release intracellular in Vero E6 cells at indicated concentrations of halofuginone after a 24 hr treatment (n=2 per dose, measured each in triplicate). Data shown as mean±S.D. Statistics performed by 1-way ANOVA (ns: p>0.05).

FIG. 30. Comparison of Halofuginone and Chloroquine treatment for inhibition of SARS-CoV-2 infection in Vero E6 cells. Authentic SARS-CoV-2 virus infection of Vero E6 cells treated with Halofuginone and chloroquine as measured by automated immunofluorescent quantification of viral nucleocapsid (red) protein. Data shown as mean±S.D. Statistics performed by 1-way ANOVA (ns: **:p≤0.01, ***:p≤0.001).

FIG. 31A and FIG. 31B. Comparison of Halofuginone enantiomer for inhibition of HS production and SARS-CoV-2 spike RBD protein binding. Effect of 500 nM halofuginone (HF) and 500 nM of the HF enantiomers 2R,3S-(+) and 2S,3R-(−) on Hep3B and its effect on cellular staining with (A) anti-HS 10E4 mAb or (B) SARS-CoV-2 spike RBD protein binding. HSase, Heparin Lyases. Data are expressed as mean±S.D. Statistics performed by 1-way ANOVA (ns: p>0.05, *:p≤0.05, **:p≤0.01, ***:p≤0.001 ****:p≤0.0001).

FIG. 32A and FIG. 32B. Halofuginone and the 2R,3S-(+) enantiomer inhibit SARS-CoV-2 infection and cell proliferation. A-B, Measurement of authentic SARS-CoV-2 infection and cell number in Vero E6 cells treated with (A) halofuginone (HF) or (B) the 2R,3S-(+) enantiomer for 24 h. Data shown as mean±S.D.

FIG. 33A and FIG. 33B. Protein content and distribution in SARS-CoV-2 proteins. (A) Proline location and kernel density estimation of proline distribution within SARS-CoV-2 proteins. Each black line indicates the location of a proline residue in the protein. The red curves show the kernel density estimation of proline distribution within a protein, which indicates the estimated probability that a proline residue will be found in each location within a protein. Kernel density estimates were calculated using the statistical software package R (see Materials and Methods). (B) Proline distribution score (see Materials and Methods) of SARS-CoV-2, SARS-CoV, MERS-CoV, HCoV-229E, HCoV-LN63, Dengue Virus (DENV), and Chikungunya virus (CIKV) viral proteins (See FIG. Table S1) as well as collagens, lysosomal proteins and proteins involved in cholesterol biosynthesis (See FIG. Table S1). Data shown as mean±S.E.M.

FIG. 34A and FIG. 34B. Borrelidin Inhibits HS production but not SARS-CoV-2 Spike RBD protein binding. (A) Effect of 500 nM halofuginone (HF), 500 nM borrelidin and 5 μM SerSA on wild-type Hep3B and its effect on cellular staining with anti-HS 10E4 mAb. or (B) Effect of borrelidin and SerSA at indicated doses on SARS-CoV-2 spike RBD protein binding in Hep3B. HSase, Heparin Lyases. Data are expressed as mean±S.D. Statistics performed by 1-way ANOVA (ns: p>0.05, **:p≤0.01, ****:p≤0.0001 and #:p≤0.0001 for HSase vs. all conditions).

FIG. 35A, FIG. 35B, and FIG. 35C. Dosing halofuginone to golden hamsters that were first infected with SARS-CoV-2 (FIG. 35A) exerted no deleterious effect on body weight (FIG. 35B), but significantly reduced viral titer by day 5 post infection, relative to uninfected hamsters (FIG. 35C).

FIG. 36: Halofuginone is a potent inhibitor of SARS-CoV-2 attachment, infection, and replication. SARS-CoV-2 spike protein interacts with cell surface heparan sulfate and angiotensin-converting enzyme 2 (ACE2) through its Receptor Binding Domain.

FIG. 37A-FIG. 37F: Oral dosing of halofuginone to hamsters infected with SARS-CoV-2 improved hamster lung pathology relative to a control group of hamsters (PBS). Halofuginone-treated hamsters exhibited significant improvement as measured by lung weight (FIG. 37A and FIG. 37B), macroscopic lung pathology (FIG. 37C), lung pathology severity (FIG. 37D), and extended lung pathology (FIG. 37E and FIG. 37F).

FIG. 38A-FIG. 38H. Oral administration of halofuginone (1 mg/kg) reduced lung fibrosis, blood clotting, and inflammation in SARS-CoV-2 infected hamsters (FIG. 38G) relative to control (PBS; FIG. 38H).

FIG. 39A and FIG. 39B. Vero E6 cells were infected with SARS-CoV-2 for 4 h, then treated over 24 h with 0-500 nM borrelidin (BN) and halofuginone (HF), and 0-10 μM SerSA. Effects on p-eIF2α and eIF2α (FIG. 39A), and on various gene expression with active and inactive enantiomers of HF (FIG. 39B), versus DMSO control, demonstrates that inhibition is not a general response of tRNA synthetase inhibition.

### DETAILED DESCRIPTION

In various embodiments, the compound administered to a subject as described in the present disclosure is halofuginone or a derivative thereof, or a pharmaceutically acceptable salt thereof. Also contemplated in various embodiments, optionally in combination with any other embodiment described herein, is the administration of febrifugine, or a pharmaceutically acceptable salt thereof, for use in the methods described herein.

In this description, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In certain embodiments, such terms refer to minimizing the spread or worsening of the disease resulting from the administration of one or more prophylactic or therapeutic agents to a patient with such a disease.

The terms “prevent,” “preventing,” and “prevention” refer to the prevention of the onset, recurrence, or spread of the disease in a patient resulting from the administration of a prophylactic or therapeutic agent.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

### Coronaviruses

The present disclosure provides in various embodiments a method for treating a subject suffering from a corona-virus, such as COVID-19, by administering halofuginone or any one or more of its constituent enantiomers to the subject. Also included in embodiments is a method of preventing a subject from contracting a coronavirus, such as COVID-19. Further embodiments contemplate treating the subject suffering from infection, lung and other organ fibrosis and inflammation related to a coronavirus.

The present disclosure relates in part to utilization of a chemical library composed of clinically approved epigenetic readers, writers, and erasers to identify critical pathways involved in heparan sulfate biosynthesis to mitigate and prevent SARS-CoV-2 infection.

Halofuginone, a coccidiostat and synthetic analog of the natural product febrifugine derived from the herb Dichroa febrifuga, has been identified as a potent inhibitor of HS biosynthesis and SARS-CoV-2 infection^{1.15}. Halofuginone reduces heparan sulfate biosynthesis in multiple cell lines, thereby limiting the number of functional SARS-CoV-2 spike protein cell surface binding sites. Additionally, halofuginone inhibited authentic SARS-CoV-2 replication post-entry. Inhibition of prolyl-tRNA synthetase (PRS) activity was responsible for both the HS-dependent and the HS-independent antiviral properties of halofuginone. These findings identify halofuginone as a translational regulator of cell surface HS biosynthesis and a potentially potent, orally bioavailable, therapeutic for the treatment and prevention of COVID-19.

Epigenetics is an emerging frontier in science and refers to changes or modifications in gene expression that are heritable independent of changes in the DNA sequence. A strong correlation exists between epigenetic modifications and a wide array of human physiological and pathophysiological processes. These epigenetic changes or modifications can occur via DNA methylation, histone modifications (acetylation and methylation), chromatin remodeling, histone variants, microRNAs, and long noncoding RNAs and function as essential regulators that remodel host chromatin, altering host transcriptional patterns and networks in a highly flexible manner. Study of these post-translational modifications (PTM) of DNA, lysine or arginine residues in histones and other proteins through methylation and acetylation has led to the discovery of three classes of epigenetic proteins. Writers are proteins that establish DNA methylation (DNA methyltransferases (DNMTs)) or add a methyl (histone methyltransferases (HMTs)) or acetyl (histone acetyltransferases (HATS)) group in histones. HMTs catalyze the transfer of up to three methyl groups to the amino group of lysine residues or up to two methyl groups to the guanidine group of an arginine residue. Erasers are epigenetic proteins that remove DNA methylation and histone deacetylases (HDACs) and histone demethylases that reverse histone acetylation and methylation. Finally, readers are proteins that bind DNA, histones/proteins containing a particular PTM that enable these chromatin processes throughout the genome to modulate transcriptional profiles. Each of the epigenetic proteins classes contributes to controlling various genes, including once relevant for the biosynthesis of HS or heparan sulfate proteoglycan (HSPG) core proteins. The fact that epigenetic modifications are reversible offers a unique opportunity to exploit targeting these epigenetic marks therapeutically to improve or even reverse disease phenotypes. Targeting the epigenetic landscape can be used as a strategy to modulate HS biosynthesis.

In the present disclosure, a chemical library composed of clinically approved epigenetic readers, writers, and erasers were used to identify critical pathways involved in heparan sulfate biosynthesis to mitigate and prevent SARS-CoV-2 infection. Halofuginone, a coccidiostat derived from the herb Dichroa febrifuga, was identified as a potent inhibitor of SARS-CoV-2 infection. Halofuginone reduces heparan sulfate biosynthesis in multiple cell lines, thereby limiting the number of functional SARS-CoV-2 spike protein cell surface binding sites. When cells were challenged with both SARS-CoV-2 pseudotype and native virus, cells pre- and post-treated with halofuginone prevented SARS-CoV-2 infection. These findings identify halofuginone as a potent epigenetic regulator of cell surface heparan sulfate biosynthesis and potentially therapeutic for the treatment of COVID-19.

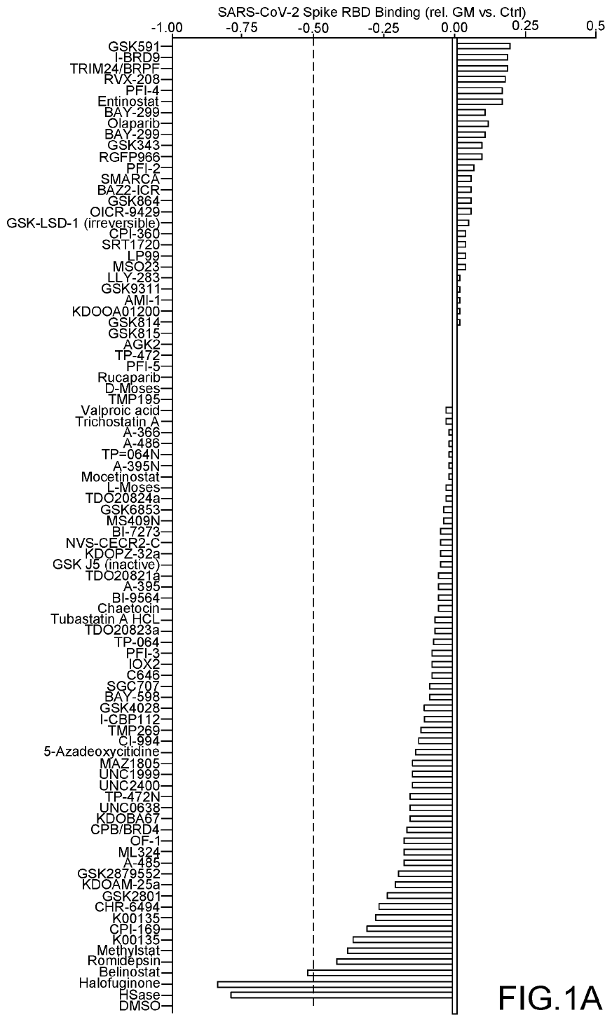

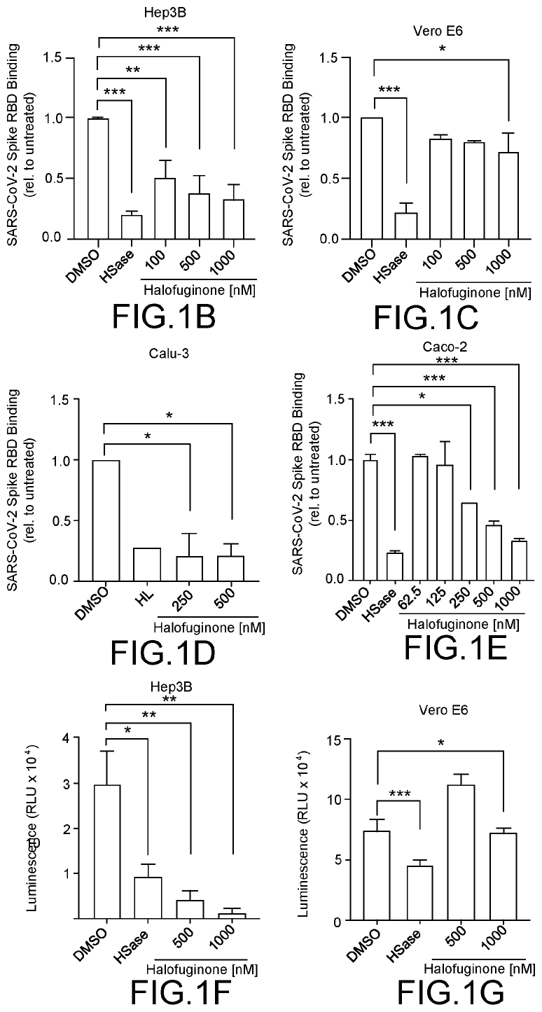

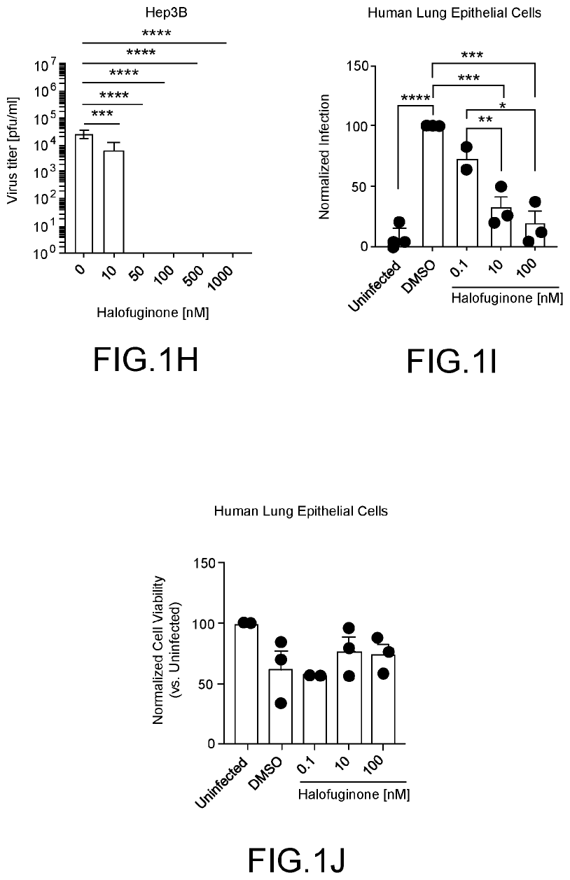

Halofuginone reduces heparan sulfate presentation. To identify epigenetic regulators that increase or decrease viral attachment, host epigenetic writers, erasers, and readers were targeted by incubating cells with a select group of small-molecule antagonists. Each compound has been confirmed to be cell-permeable and stable in cells and selectively and potently antagonize specific chromatin regulatory proteins or domains, including protein-specific acetyltransferases and methyltransferases demethylases, deacetylase and bromodomains. Moreover, this library contains chemical matched probes that are structurally similar to the active probe but do not affect epigenetic protein modulators. Of the 112 compounds, 8 reduced the amount of bound 10E4, a monoclonal antibody that whose antigen is heparan sulfate, by at least two-fold while 14 increased the amount of 10E4 stained by two-fold. The compounds that reduced heparan sulfate presentation included 5-Azadeoxycitidine, OF-1, 5-Iodotubercidin, CPB/BRD4, KD0BA67, A-485, 5-Azacytidine, and halofuginone (MAZ1392) while UNC0638 and Romidepsin increased the total amount of heparan sulfate (FIG. S1). By the time these studies were underway, the SARS-CoV-2 virus was being investigated for its potential to engage heparan sulfate as a host attachment factor. To identify host epigenetic regulators of SARS-CoV-2, we perform the compound screen using recombinant SARS-CoV-2 spike protein. Interestingly, only two compounds, reduced spike protein by more than two-fold, halofuginone, and belinostat (FIG. 1A). Halofuginone targets the prolyl-tRNA synthetase, and belinostat is a hydroxamic acid-type histone deacetylase (HDAC) inhibitor. While halofuginone had the most significant inhibition of spike protein binding and 10E4 staining, belinostat increased 10E4 staining and reduced spike protein binding.

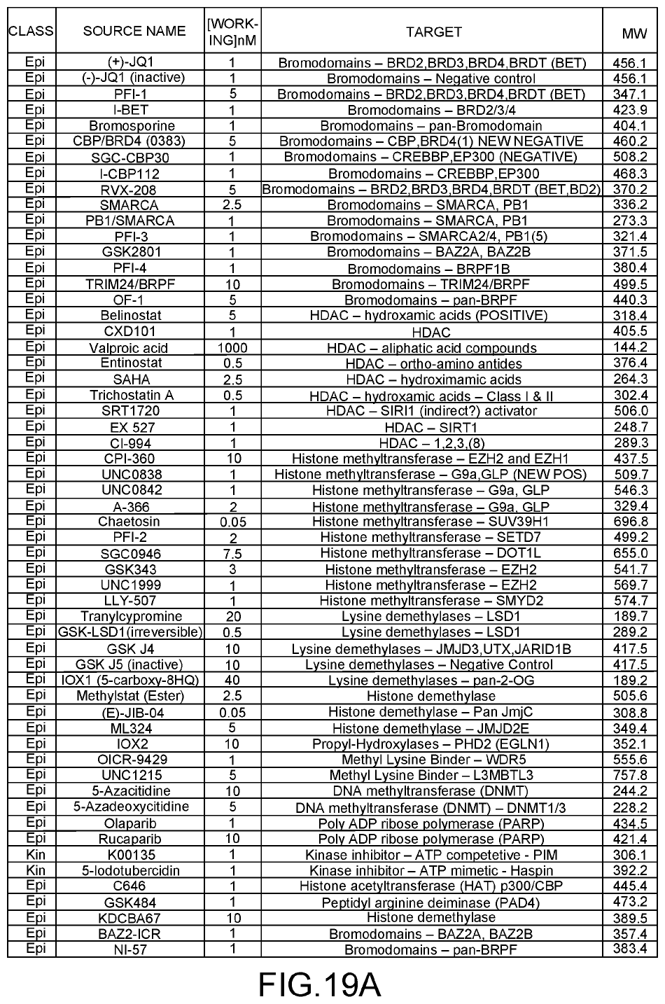

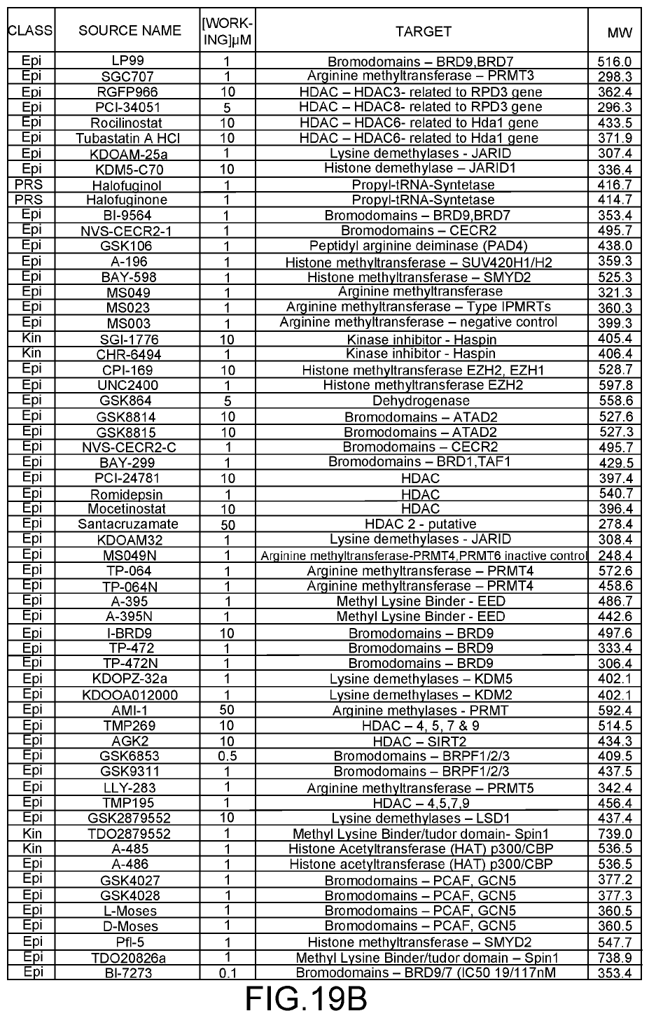

More specifically, per some embodiments, identified herein, are potential agents that attenuate SARS-CoV-2 spike protein binding by screening a library of small-molecule antagonists of host epigenetic regulators and protein translation elements of the prolyl-tRNA synthetase complex, along with their chemically matched inactive analogs¹⁶⁻¹⁸. The compound library has been successfully used to investigate targets mediating anti-inflammatory or anti-proliferative effects in a variety of biological contexts¹⁶⁻¹⁸ and importantly, contains several FDA-approved drugs, which may facilitate rapid deployment for treatment of COVID-19 patients. Hep3B human hepatoma cells were treated with the compound library at the indicated concentrations (FIG. 1A, FIG. 19A, and FIG. 19B) for 18 h, and binding of SARS-CoV-2 spike protein was assessed using recombinant receptor binding domain (RBD; from isolate Wuhan 20) in flow cytometry (FIG. 1A). As a positive control cells were treated with a mixture of heparin lyases, which digest HS chains³. Class 1 histone deacetylase inhibitors, such as Romidepsin (1 μM) or Belinostat (5 μM), decreased SARS-CoV-2 RBD binding by ˜50% compared to excipient control (DMSO; FIG. 1A). Halofuginone (1 μM) reduced binding by 84%, a level of reduction similar to the impact of heparin lyase treatment (FIG. 1A). Halofuginone targets the prolyl-tRNA synthetase (PRS) active site of the human glutamyl-prolyl tRNA synthetase (EPRS) and has been used in clinical and preclinical studies to treat fibrotic disease and to attenuate hyperinflammation^{1,19}.

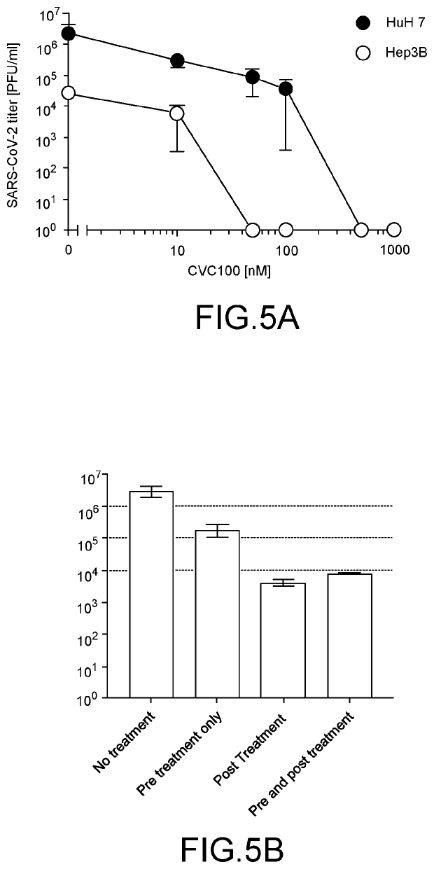

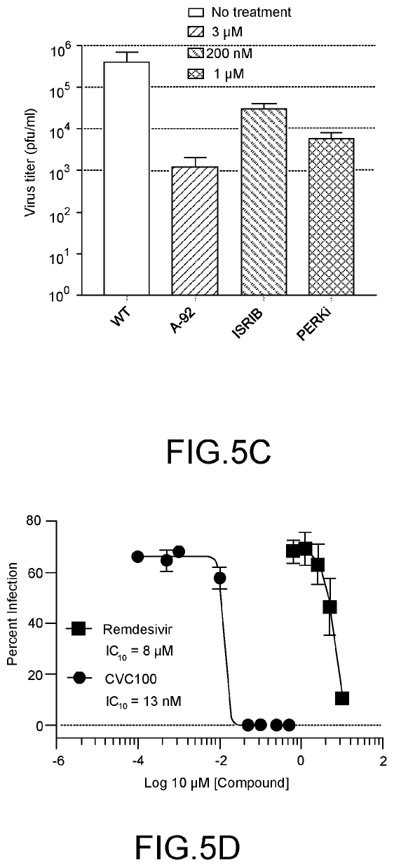

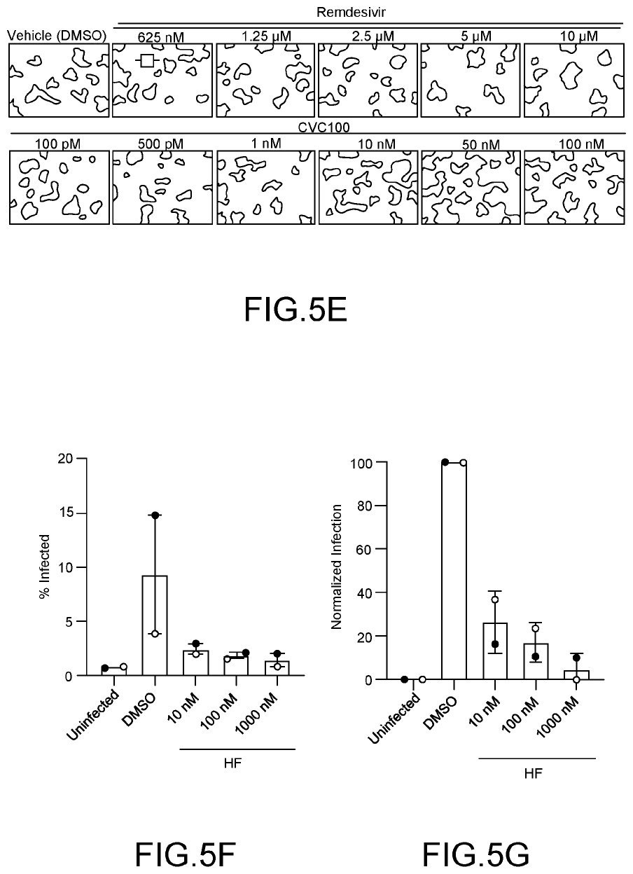

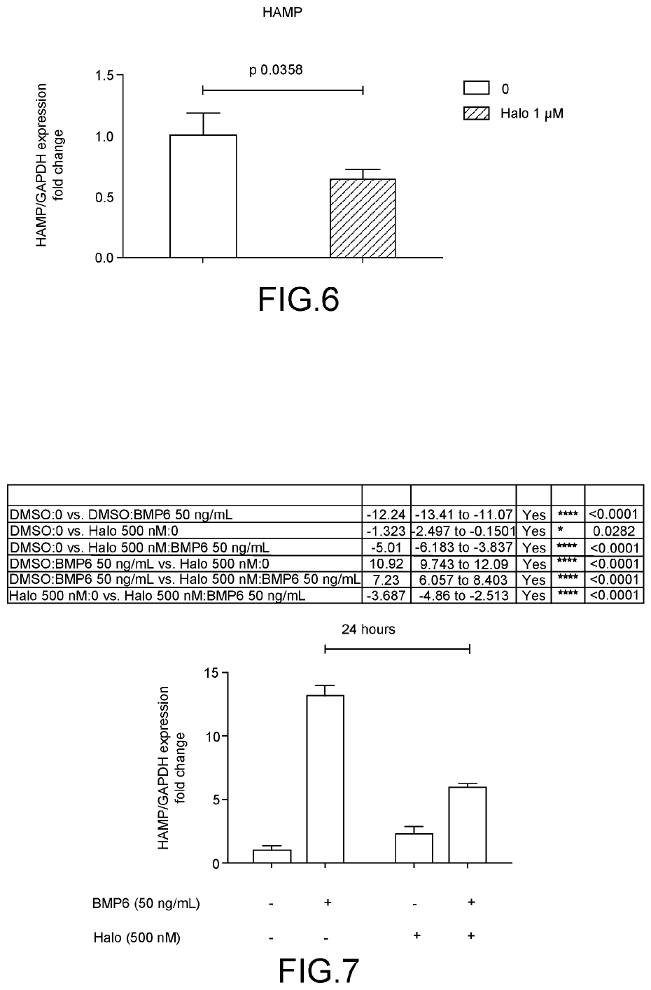

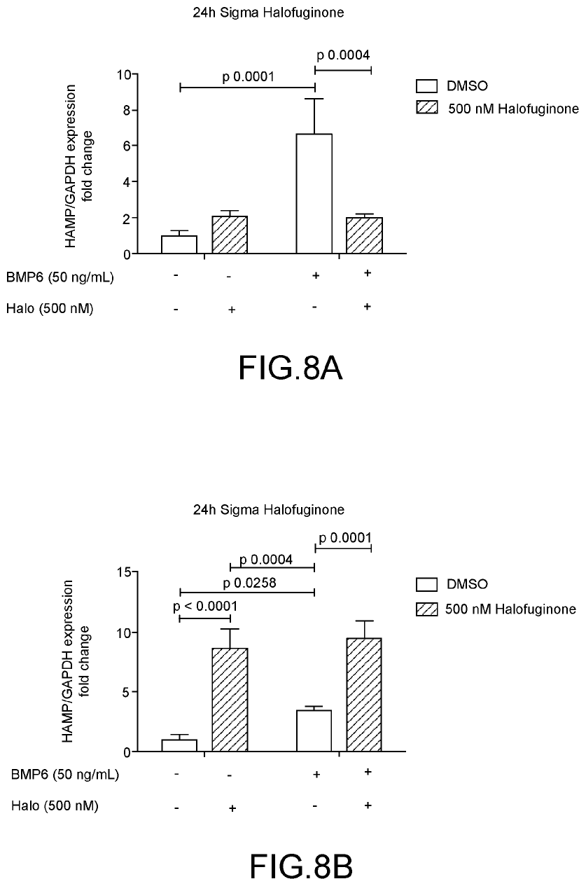

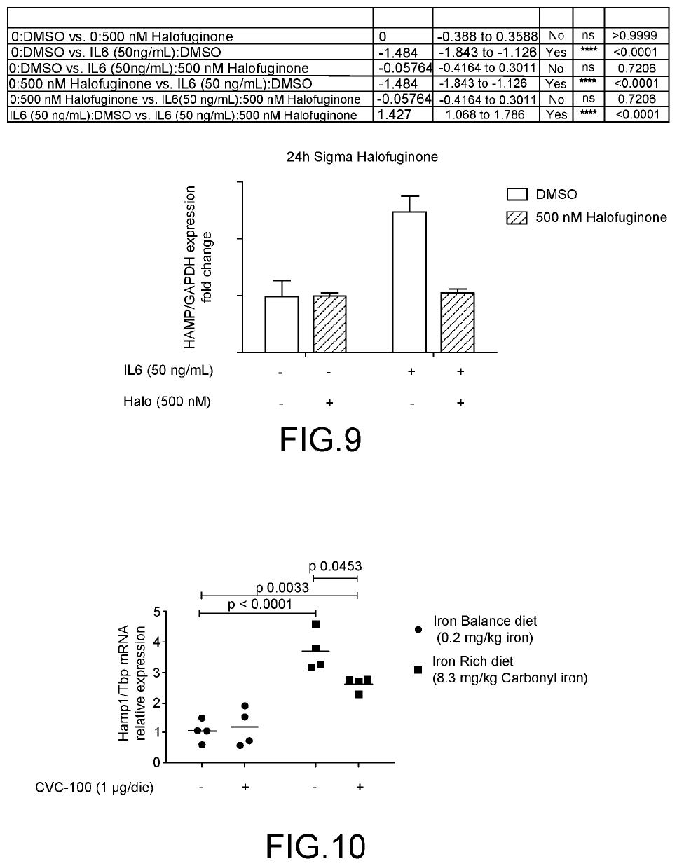

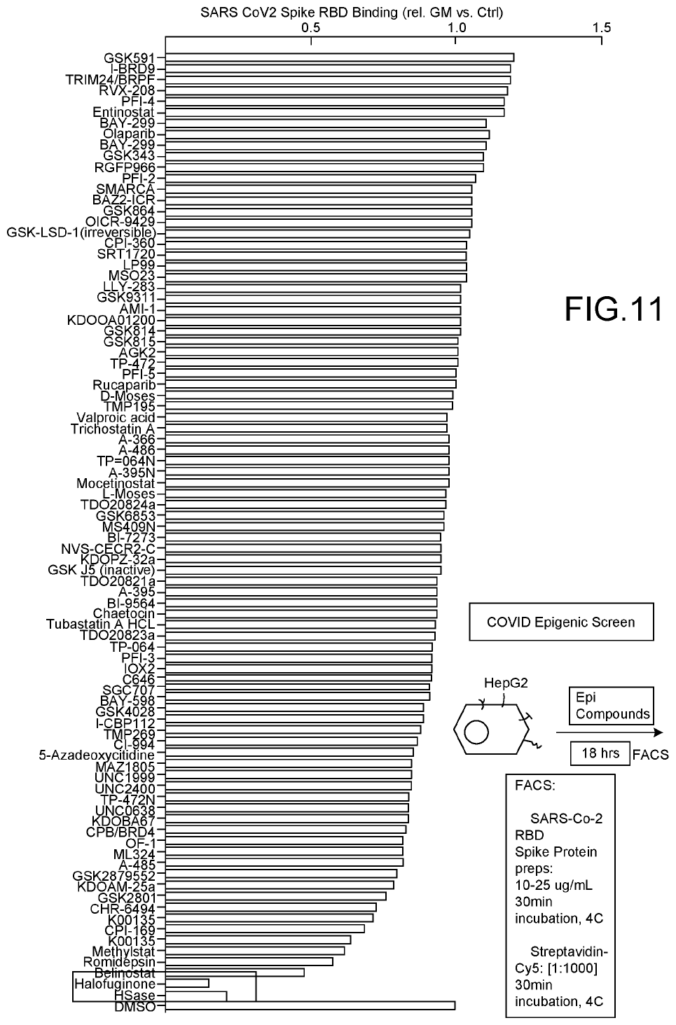

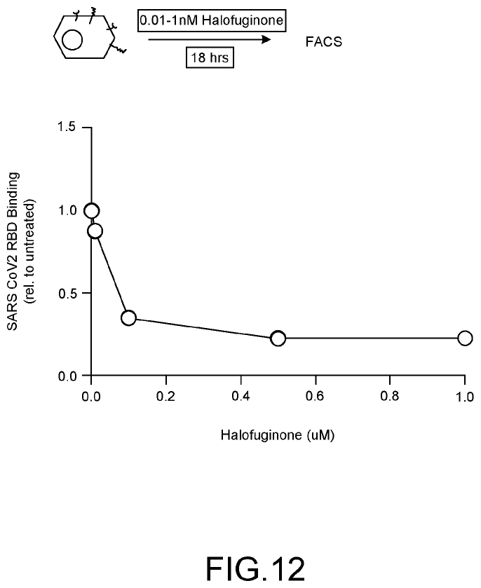

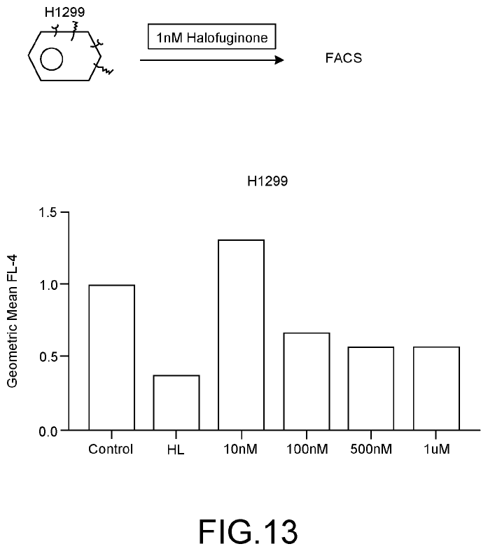

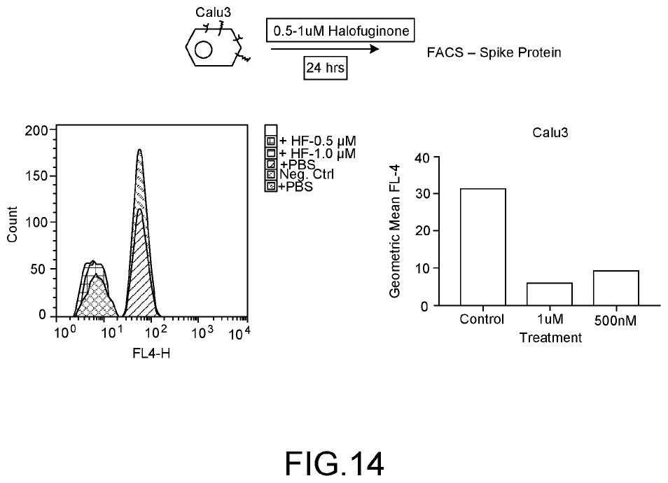

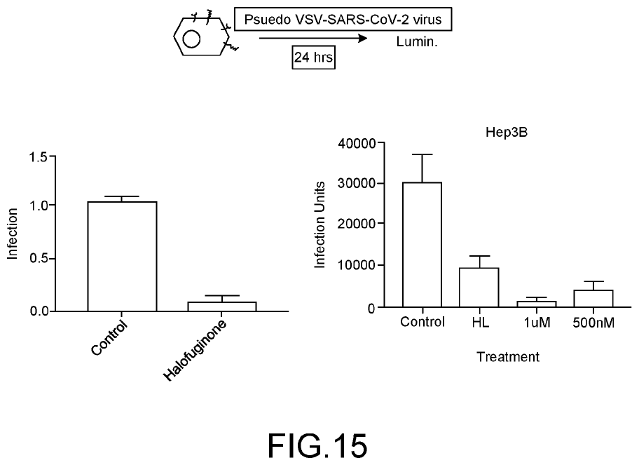

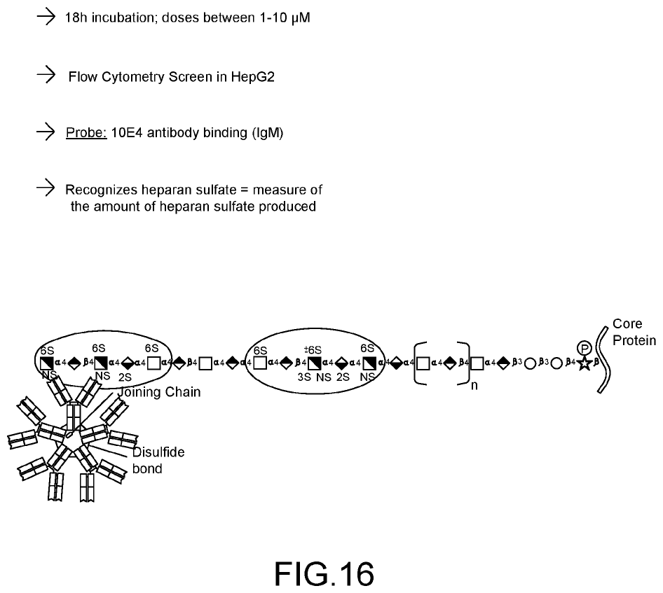

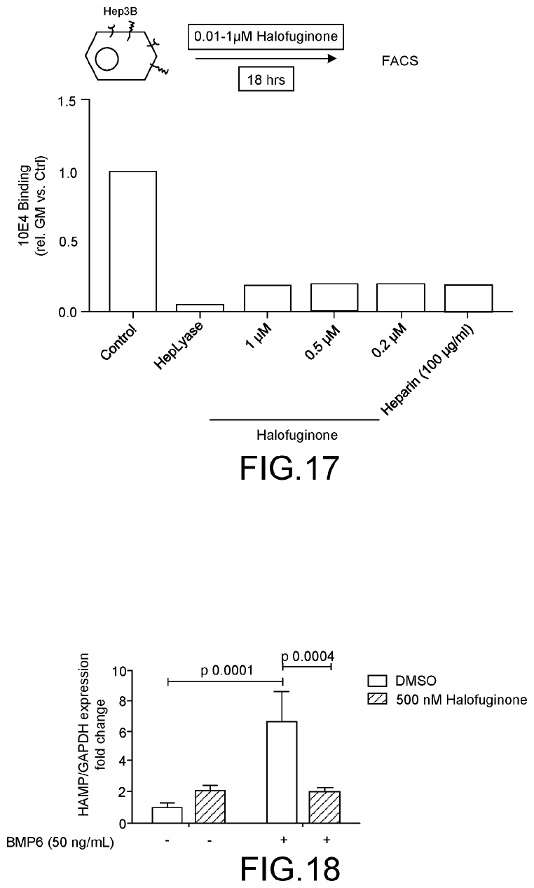

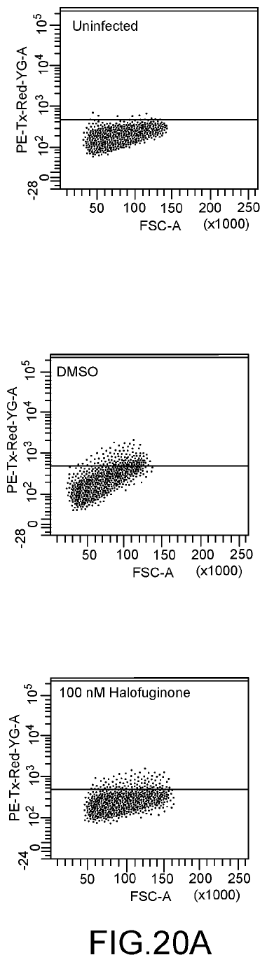

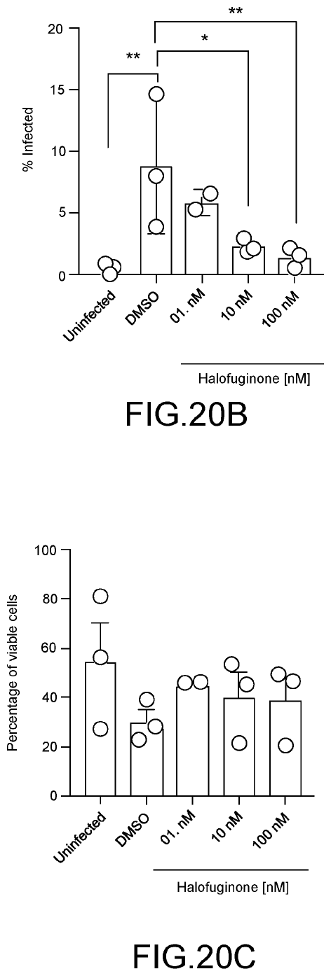

Vero E6 African green monkey kidney epithelial cells, Caco-2 human epithelial colorectal adenocarcinoma cells, and Calu-3 human epithelial lung adenocarcinoma cells were then treated with halofuginone. Halofuginone reduced spike RBD protein binding to Hep3B, Calu-3 and Caco-2 but showed very modest effects in Vero E6 cells (FIG. 1B-E). The degree of inhibition was greatest in the Calu-3 line (5-fold reduction in spike RBD binding, FIG. 1E). To test the effect of halofuginone on the binding of spike protein in a more native presentation, we examined the ability halofuginone to inhibit infection of SARS-CoV-2 spike protein pseudotyped VSV in Hep3B cells. Halofuginone inhibited infection by up to about 30-fold in a dose-dependent manner (FIG. 1F). Consistent with the inability of halofuginone to reduce spike RBD binding to Vero E6 cells (FIG. 1C), no inhibition of infection was seen in these cells (FIG. 1G). Next halofuginone was tested to see if it inhibits the infection of authentic SARS-CoV-2 in Hep3B. Cells were treated with halofuginone at different doses for 24 h prior to and during infection (MOI of 0.1) (FIG. 5A-B)²⁰. The culture medium was collected, and the presence of virus was measured by plaque assays in Vero E6 cells. No infectious SARS-CoV-2 could be detected in the supernatants of Hep3B cells treated with halofuginone at doses greater than 50 nM (FIG. 1H). To explore the effect of halofuginone on SARS-CoV-2 infection in a more clinically relevant model, primary human bronchial epithelial cells were grown at an air-liquid interface and infected them with authentic SARS-CoV-2 virus with and without halofuginone treatment. Halofuginone significantly reduced the number of SARS-CoV-2 infected cells at both 10 nM and 100 nM without affecting cell viability (FIG. 1I-J & FIGS. 20A and 20B).

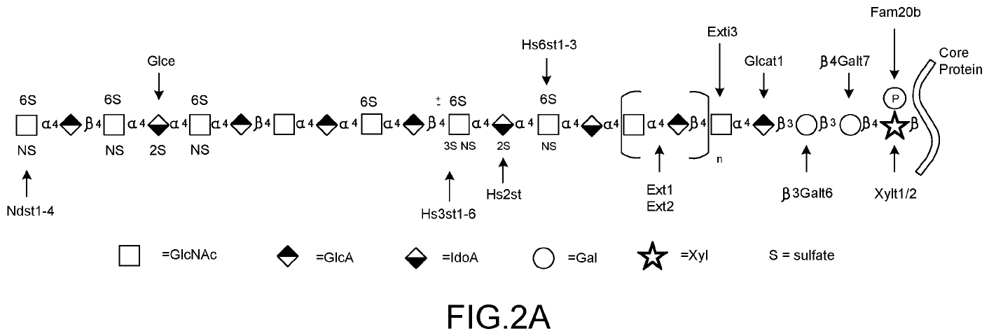

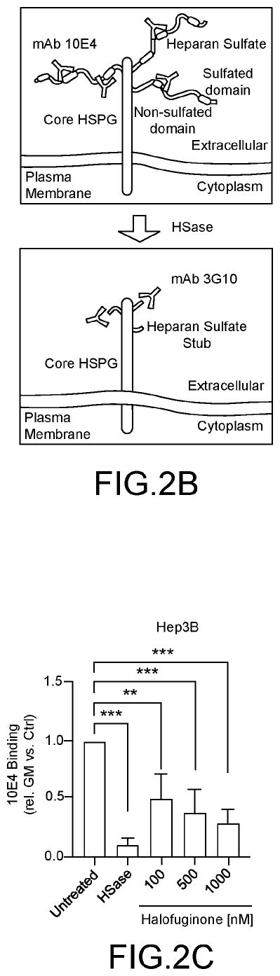

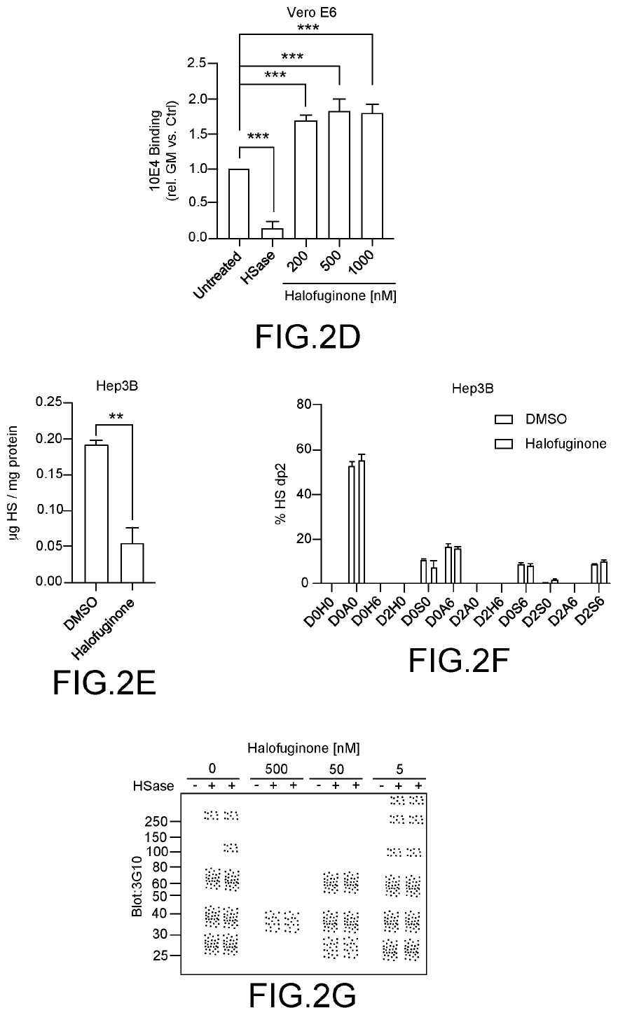

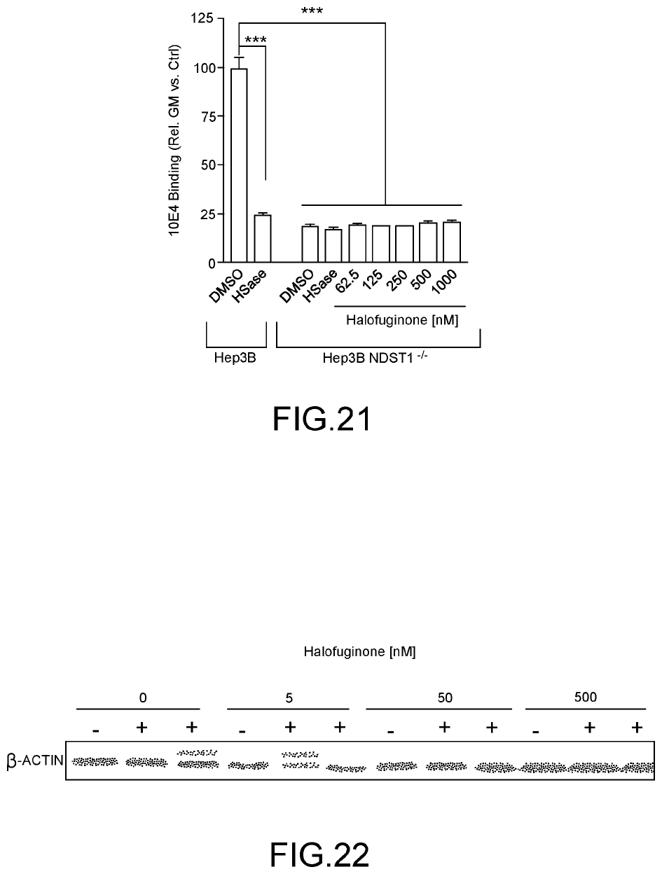

Halofuginone Inhibits Heparan Sulfate Biosynthesis. The binding of SARS-CoV-2 spike protein to cells is HS-dependent³. HS is a linear polysaccharide attached to serine residues in HS proteoglycans (HSPGs)²¹. The HS polysaccharides consist of alternating residues of N-acetylated or N-sulfated glucosamine (GlcNAc or GlcNS) and either glucuronic acid (GlcA) or iduronic acid (IdoA) (FIG. 2A). Hence, halofuginone was tested to see whether it reduces spike protein binding by reducing HS presentation at the cell surface. Hep3B and Vero E6 cells were treated for 18 h with halofuginone and the effects on cellular HS were evaluated using the monoclonal antibody (mAb) (10E4) that recognizes a common epitope in HS (FIG. 2B & FIG. 21). Halofuginone dose-dependently reduced 10E4 binding in Hep3B (FIG. 2C), whereas 10E4 binding increased in Vero E6 upon halofuginone treatment (FIG. 2D). This directly correlates with the level of spike RBD binding and S protein pseudotyped virus infection in these cells (FIG. 1B-C). This finding suggests that halofuginone inhibits spike protein binding and SARS-CoV-2 viral attachment by altering cell surface HS content. Analysis of Hep3B cells showed that 0.5 μM halofuginone reduced cell surface HS in Hep3B cells by ˜4-fold (FIG. 2E). No difference was seen in the disaccharide composition following halofuginone treatment, suggesting that halofuginone affects total HS synthesis but does not alter HS specific sulfation (FIG. 2F). To test if the observed decrease in total HS was due to a decrease in availability of HSPG core proteins to carry HS, treated Hep3B cells were lysed, and the lysates were run on SDS-PAGE and stained using an mAb (3G10) that recognizes a neo-epitope remaining on the proteoglycans (FIG. 2B,G)²². The analysis revealed that halofuginone reduced the expression of core HSPGs in a dose-dependent manner (FIG. 2G & FIG. 22). These data suggest that halofuginone inhibits HS-mediated binding of the SARS-CoV-2 spike protein to cells by inhibiting the expression of HSPGs.

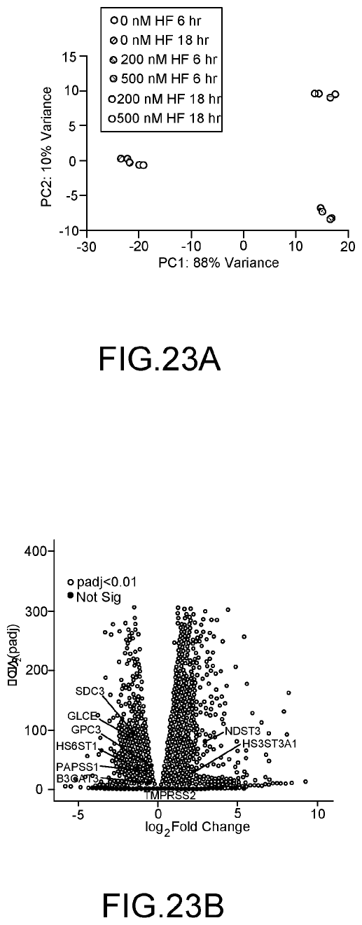

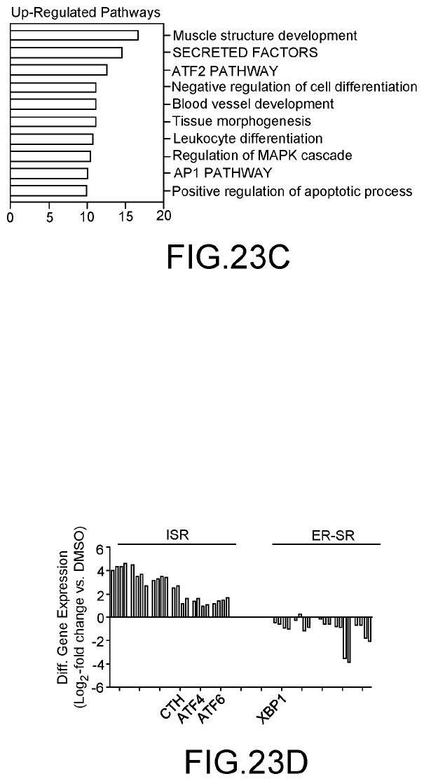

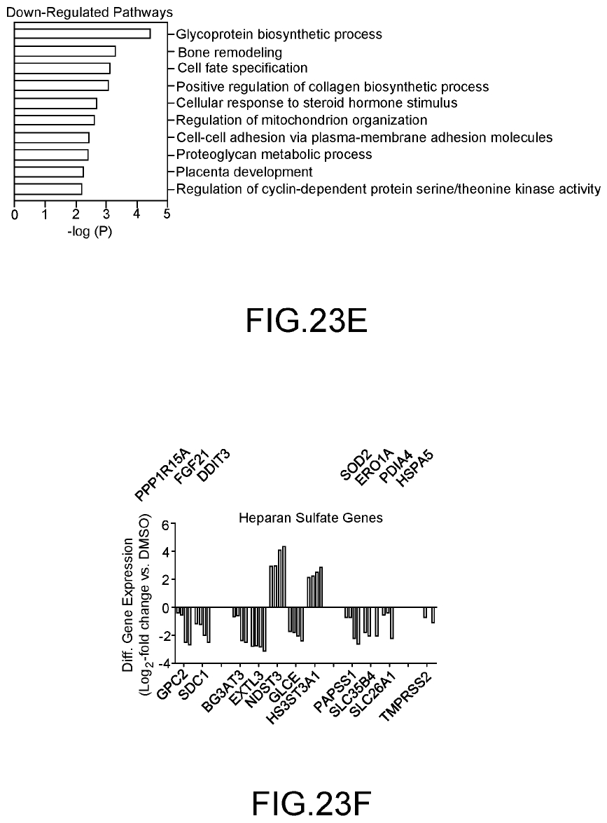

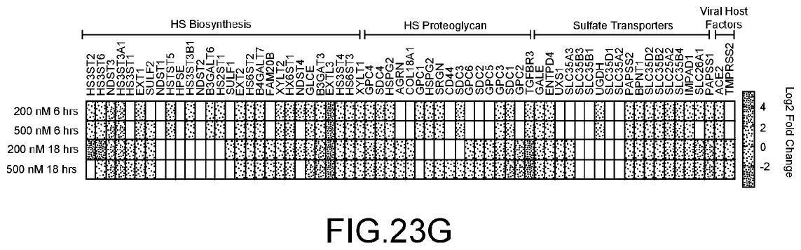

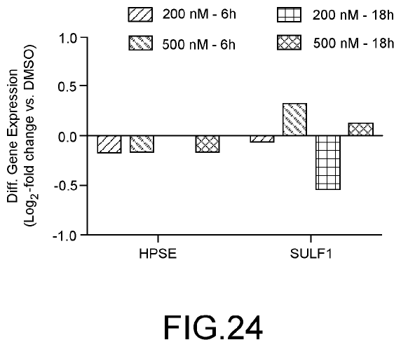

To broadly examine the effects of halofuginone treatment, Hep3B cells were treated with vehicle or halofuginone at 200 or 500 nM for 6 h and 18 h and processed for RNA-sequencing (RNA-Seq). Halofuginone profoundly impacted the transcriptome (˜2700 differentially expressed genes), which increased with longer incubation time (FIGS. 23A and 23B). Principal component analysis clearly segregated the response with respect to the duration of treatment (FIG. 23A). Analysis of halofuginone upregulated genes did not identify common pathways associated with antiviral or inflammatory responses (FIGS. 23C-23D)²³. Additionally, no significant difference in expression of host factors exploited by SARS-CoV-2, such as TMPRSS2 and ACE2, were observed (FIGS. 23F and 23G). Gene annotation analysis of downregulated genes at 6 h showed that 200 nM halofuginone altered the expression of genes involved in glycoprotein biosynthesis and proteoglycan metabolic processes (FIG. 23E)²³. A select group of core HSPGs were downregulated at the mRNA level (FIGS. 23F and 23G). In particular, HSPGs GPC2 and SDC1 were significantly downregulated in conjunction with the HS biosynthetic enzymes B3GAT3 and EXTL3 (FIGS. 23F, 23G, and 24). Taken together, the data demonstrates that halofuginone suppresses the expression of proteins involved in HS and HSPG production.

Halofuginone Inhibits SARS-CoV-2 Spike protein binding and pseudovirus infection. These data suggest that these compounds functionally target different pathways. Halofuginone was the most potent inhibitor of recombinant SARS-CoV-2 spike protein binding and heparan sulfate biosynthesis. The heparan sulfate cellular dependence of spike protein binding to a variety of cell types was tested, including Hep3b, VeroE6, Caco-2, and Calu-3 cells. Each cell line is permissive to SARS-CoV-2 infection and can engage recombinant SARS-CoV-2 spike protein. Biotinylated Spike protein bound to each cell to a variable degree, spike protein binding was heparan sulfate dependent as cell treated with bacterial heparan sulfate lyases ablated spike protein binding (FIG. 1B-E). These data confirm that spike protein binding is heparan sulfate dependent.

Next, it was tested whether halofuginone can recapitulate the reduction in spike cell surface protein binding. With exception to Vero E6 cells (an African Green Monkey Kidney cell line) (FIG. 1C), halofuginone dose-dependently inhibited spike protein binding in all other human cell lines such as Hep3B, Calu-3, and Caco-2 cells (FIG. 1B, D-E). As recombinant spike protein does not recapitulate the multivalent nature of a virion, we tested whether halofuginone may inhibit infection of SARS-CoV-2 pseudovirus. Halofuginone dose-dependently inhibited SARS-CoV-2 pseudovirus infection in Hep3B (FIG. 1F). Halofuginone did not inhibit pseudovirus attachment and invasion in Vero E6 cells (FIG. 1G), which is in line with spike protein binding data (FIG. 1C). Halofuginone dose-dependently reduced the presence of cell surface heparan sulfate Hep3B (FIG. 2A). However, halofuginone in Vero E6 cells did not significantly alter heparan sulfate production and presentation (FIG. 2B), suggesting that halofuginone inhibits spike protein binding SARS-CoV-2 attachment by reducing cell surface heparan sulfate.

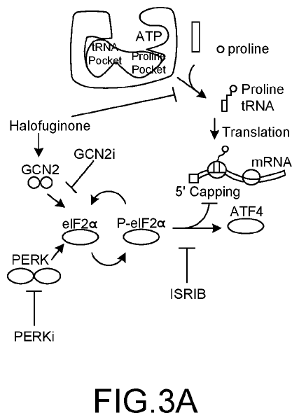

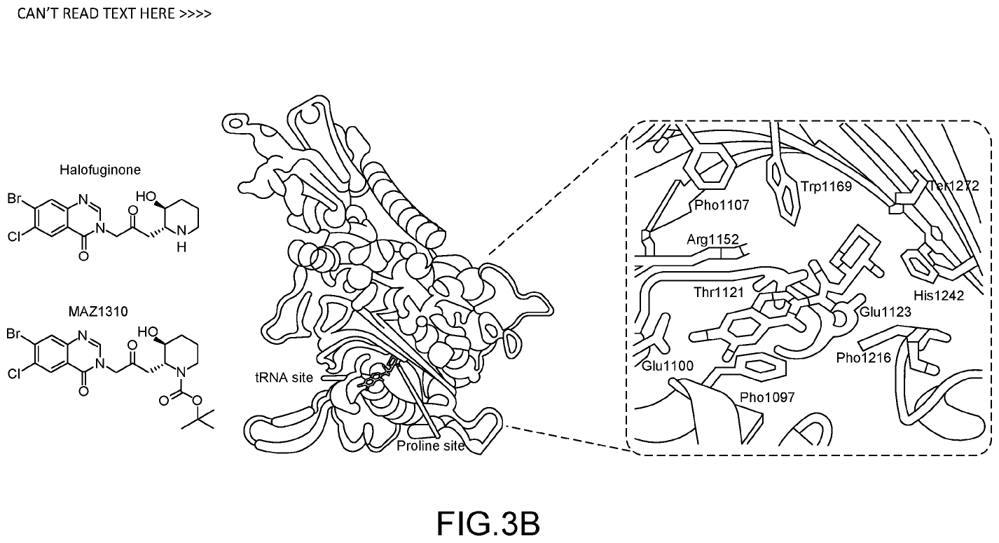

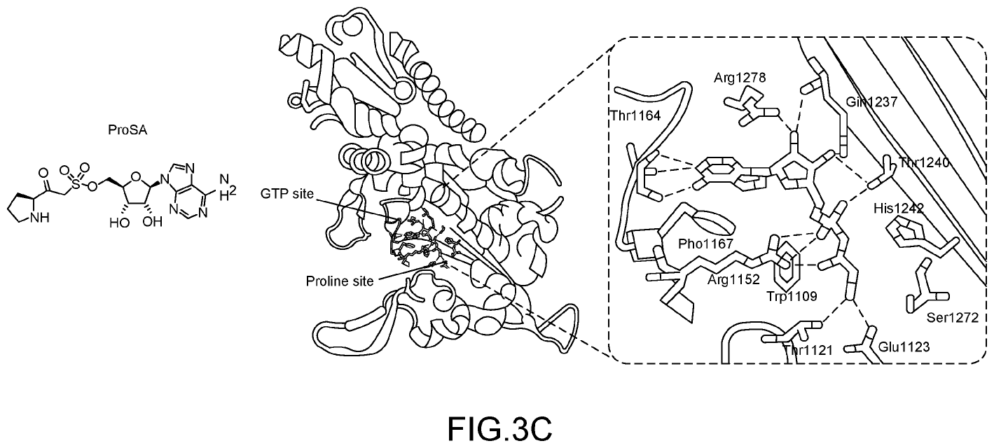

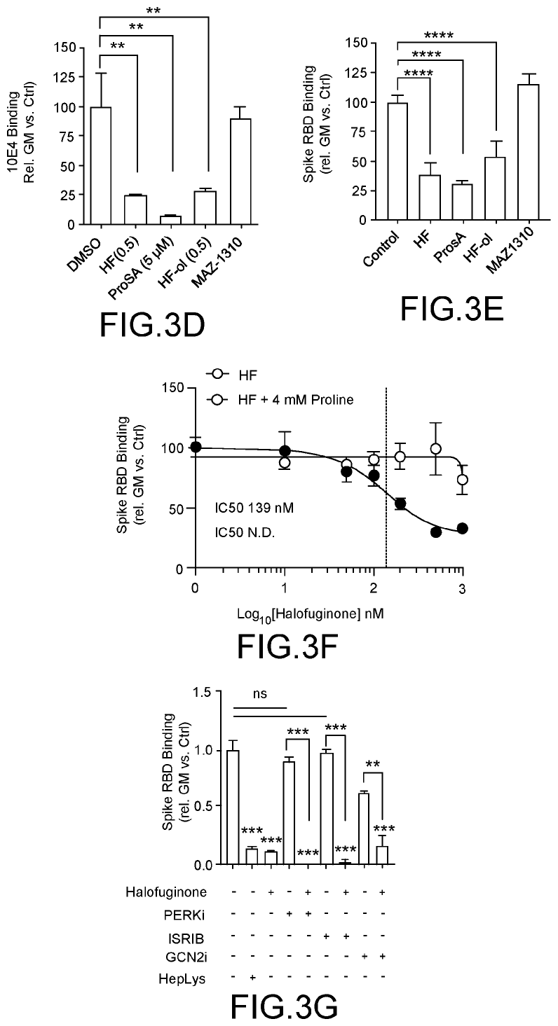

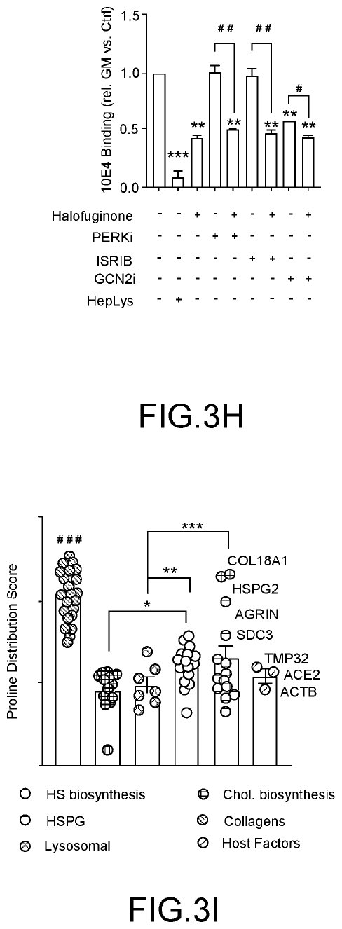

Halofuginone Inhibition of heparan sulfate presentation and Spike protein binding is independent of integrated stress response activation. Halofuginone inhibits the Prolyl-tRNA Synthetase (FIG. 3A), which will block the RNA translation of proteins enriched for proline. This event will, in turn, also activate the integrated stress response as halofuginone activates GCN2 to phosphorylate the eukaryotic transcription initiation factor 2a (eIF2a), which will activate the transcription factor ATF4 and also inhibit 5′cap-mediated RNA translation (FIG. 3A). It was tested whether the halofuginone induced reduction in heparan sulfate biosynthesis evaluates was a consequence of the integrated stress response or inactivation of Prolyl-tRNA Synthetase (PRS)-mediated RNA translation. Halofuginone was co-incubated in the presence of general inhibitors of the integrated stress response, ISRIB, an inhibitor of PREK-mediated activation of the integrated stress response (PERKi; GSK2606414) or an inhibitor of GCN2 (GCN2i; GCN2-IN-1). It was evaluated whether the inhibitors can prevent the halofuginone-mediated reduction in heparan sulfate presentation via 10E4 binding and Spike protein binding (FIG. 3B). The GCN2i reduced in heparan sulfate production (FIG. 3B) and Spike protein binding (FIG. 3C) independent of halofuginone, suggesting a regulatory role for GCN2 in heparan sulfate production regulation independent of the integrated stress response. Both the PERKi and ISRIB were unable to attenuate the halofuginone-induced reduction in heparan sulfate production and Spike protein binding, suggesting that this effect is independent of the integrated stress response activation (FIG. 3B-C).

Halofuginone Inhibition of Spike Protein Binding to Heparan Sulfate is Independent of the Canonical Integrated Stress Response. Aminoacyl-tRNA-synthetases (AARS) catalyze the ATP-dependent synthesis of amino-acylated tRNAs via a two-step reaction involving an aminoacyl-adenylate intermediate with subsequent transfer to the cognate tRNAs²⁴. Halofuginone and chemically unrelated compounds like prolyl-sulfamoyl adenosine (ProSA), specifically and potently inhibit human prolyl-tRNA synthetase (PRS) by blocking distinct portions of their ligand binding pockets (FIG. 3A-C)^{15.25}. PRS inhibition can specifically suppress translation of proteins, such as collagens, that are enriched in prolines while having minimal effects on general protein synthesis¹⁵. However, PRS inhibition can also lead to GCN2-mediated activation of the Integrated Stress Response (ISR). GCN2 (gene symbol EIF2AK4) senses uncharged tRNAs and phosphorylates the eukaryotic transcription initiation factor 2α (eIF2α), leading to a general reduction in 5′cap-mediated RNA translation and selective translation of the eukaryotic transcription factor ATF4 and its target genes (FIG. 3A)²⁶. These genes contain structural features in their 5′UTR allowing for selective translation in the presence of Ser51 phosphorylated eIF2α. The complexity and transient nature of the transcriptional and translational changes mediated by different eIF2α kinases of the ISR allows a cell to adapt and resolve various stress situations including amino acid starvation or unfolded protein stress²⁶.

Halofuginone induced a general ATF4-mediated ISR but did not activate the unfolded protein-induced ER stress response, as reported previously (FIG. 23D)²⁷. To date, alterations in HS biosynthesis have not been identified as a hallmark of the ISR. To better understand the relationship between PRS inhibition and HS expression, related PRS inhibitor analogs were tested to deconstruct the PRS pathway in relation to HS biosynthesis and spike RBD binding. Treatment with the non-cleavable and highly selective prolyl-AMP substrate analog ProSA at 50 μM prevented HS presentation and spike RBD binding to similar levels as treatment with halofuginone (500 nM), as illustrated by mAb 10E4 stain (FIG. 3C-D)²⁸. Additionally, halofuginol (HFol), a halofuginone derivative that inhibits PRS, significantly decreased 10E4 and spike RBD binding while the MAZ1310 negative control compound, had no effect (FIG. 3D)^{15,25}. Collectively, the data demonstrates that PRS inhibition is sufficient to reduce HS biosynthesis and spike RBD binding (FIG. 3D-E). Halofuginone competes with proline for the PRS active site¹⁵. Addition of excess proline to the media (4 mM) of Hep3B cells prevented the ability of halofuginone to inhibit spike RED protein binding to the cells (FIG. 3F).

To determine if halofuginone suppresses HS biosynthesis by activating the ISR, halofuginone was co-incubated in the presence of a general inhibitor of the ISR, ISRIB (Integrated Stress Response inhibitor), selective eIF2α kinase inhibitors GCN2-IN-1 (GCN2i) targeting GCN2 (general control nonderepressible 2), or GSK2606414 (PERKi) targeting eIF2α kinase 3 (eIF2AK3), also known as protein kinase R-like endoplasmic reticulum kinase (PERK) (FIG. 3A)^{26,29}. Neither PERKi nor ISRIB affected 10E4 or spike RBD binding, and neither had an effect on halofuginone inhibition (FIG. 3G-H). In contrast, GCN2i reduced 10E4 and spike RBD binding almost to the same extent as halofuginone activity (FIG. 3G-H), suggesting a role for GCN2 in the regulation of HS biosynthesis independent of the ISR. Neither GCN2-IN-1, GSK2606414, nor ISRIB reversed the halofuginone induced reduction in 10E4 or spike protein binding, suggesting that halofuginone does not suppress HS biosynthesis and spike protein binding by activating the ISR (FIG. 3G-H).

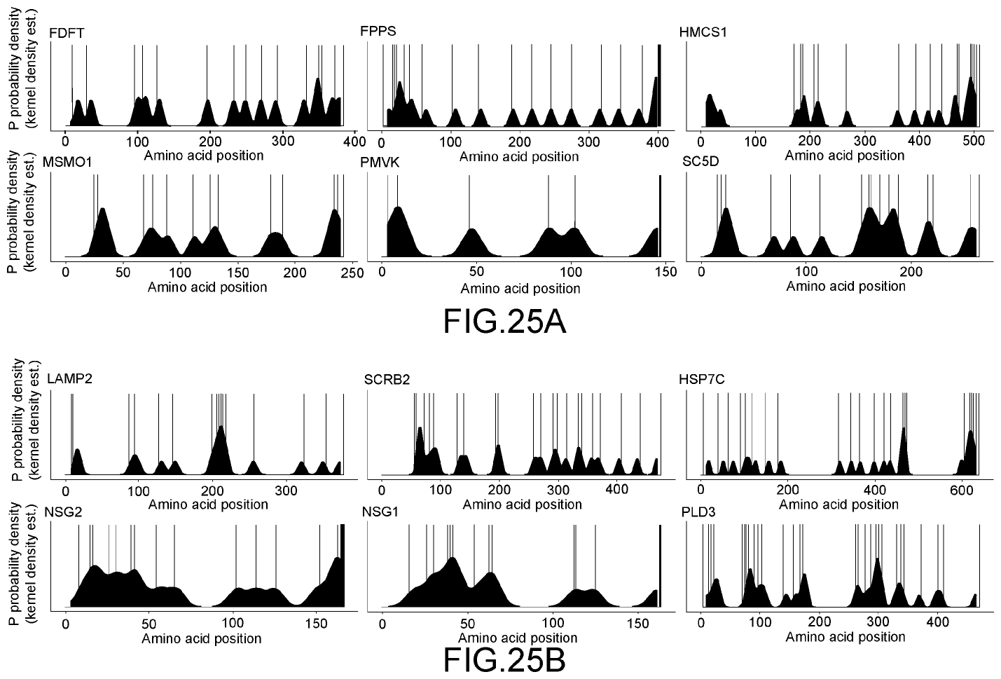

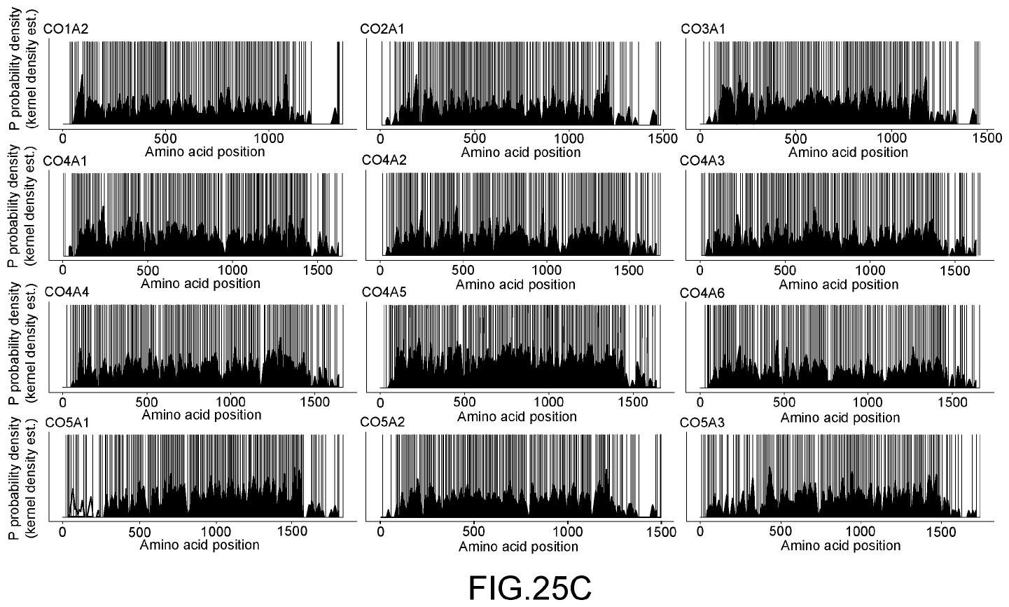

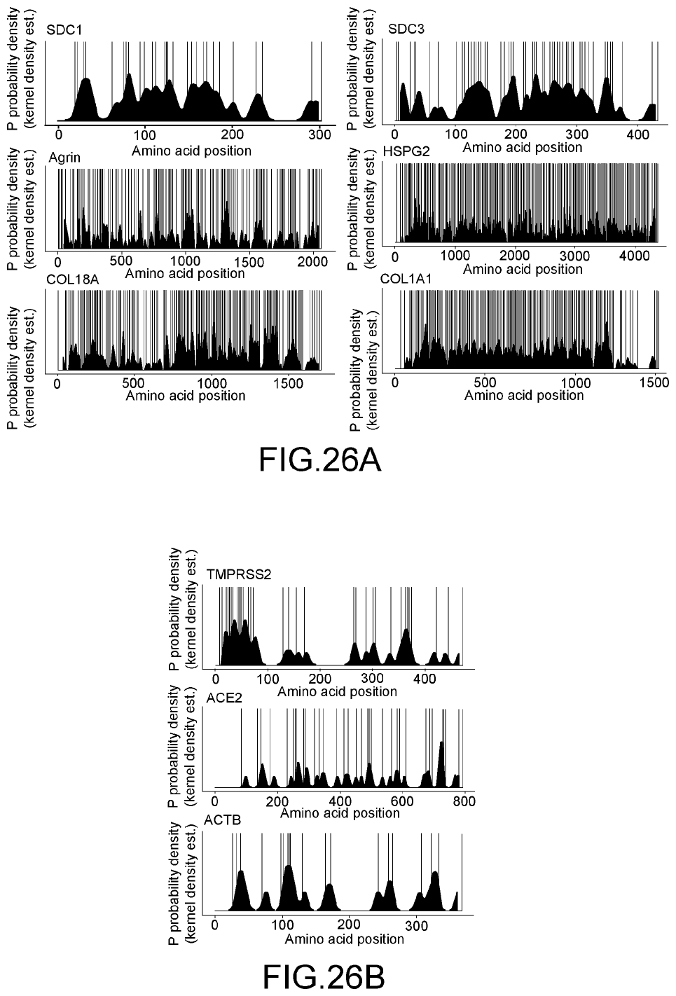

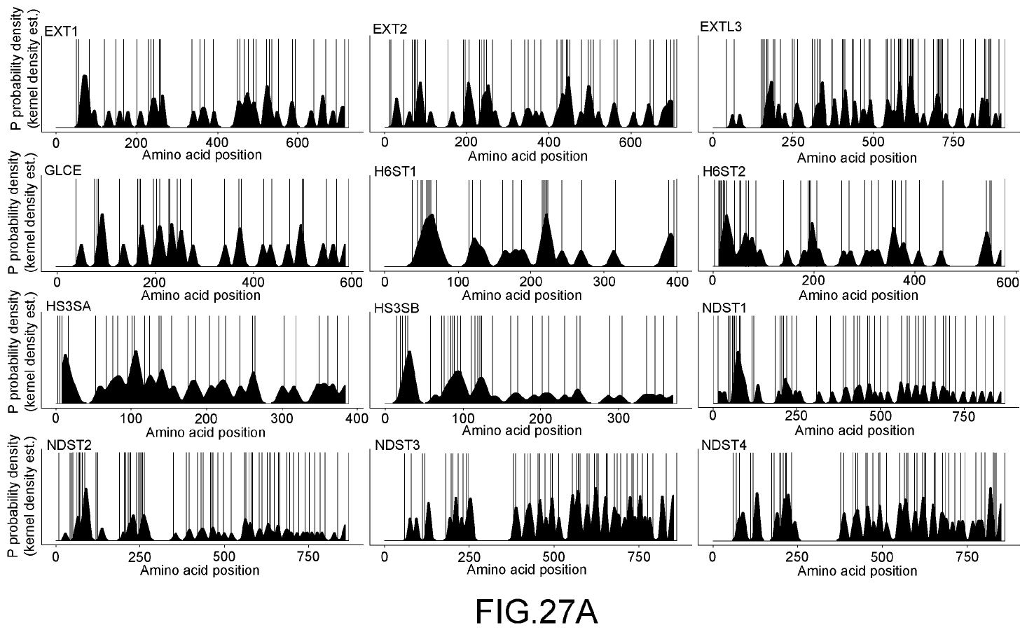

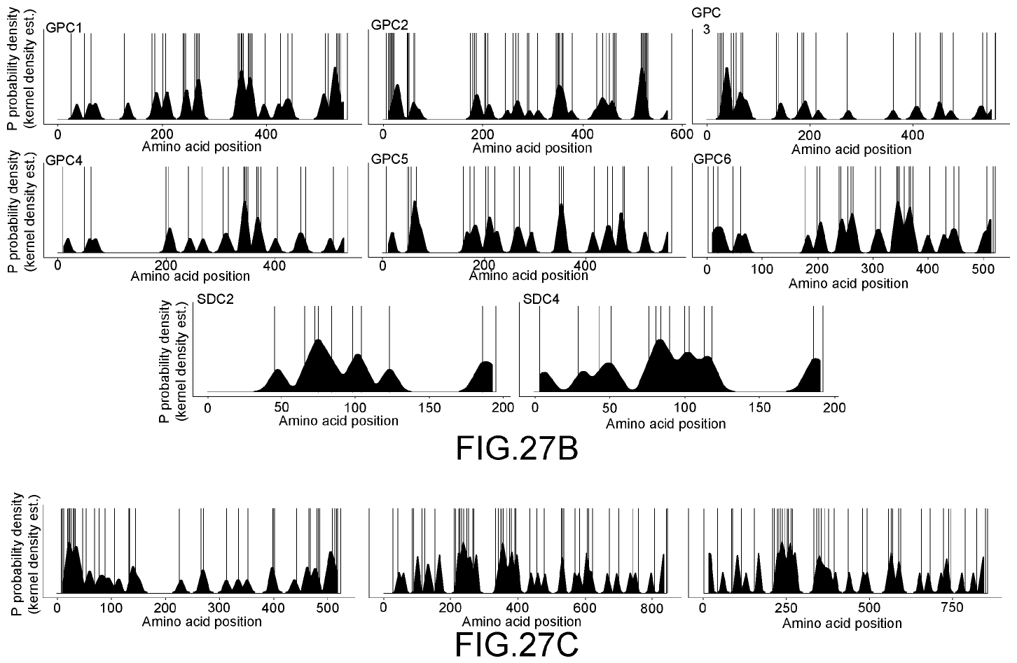

PRS inhibitors can selectively modulate the translational efficiency of proline-rich proteins, such as collagens (FIGS. 25A -25C). Interestingly, HSPGs, such as agrin, perlecan, collagen 18 and syndecans 1 and 3, are relatively proline-rich compared to other proteins, such as TMPRSS2, ACE2 and lysosomal or cholesterol biosynthetic proteins (FIGS. 3I and 25-27). These observations suggest that inhibition of prolyl-tRNA charging could impair production of key membrane and extracellular matrix HSPGs (FIGS. 3I and 2G). Together, these data suggest that PRS inhibitors, such as halofuginone, inhibit HS and proteoglycan expression both at the translational and transcriptional level.

Halofuginone Inhibition of heparan sulfate production and spike protein binding is dependent on PRS translation inhibition. Based on the above experiments, it was evaluated whether the PRS inhibition and ensuing attenuation of production in proline-rich proteins can explain the halofuginone induced reduction in heparan sulfate biosynthesis. Therefore, Hep3B cells were treated with a halofuginone analog (HF-ol), other classes of PRS inhibitors, and t-RNA-synthetase inhibitors as well as respective negative controls (OBT-J and MAZ1310). At 1 μM, all the tested PRS and t-RNA-synthetase inhibitors reduced heparan sulfate presentation, and Spike protein binding to a similar extent as halofuginone (FIG. 3D). Only ProSA, a activate proline-AMP donor inhibitor, was unable to inhibit heparan sulfate inhibition and Spike protein binding, although this is due to the reduced permeability of the compound due to its polarity. PRS inhibition will mainly target proline-enriched proteins such as Collagen A1 (ColA1) (FIG. 3D). A bioinformatic evaluation revealed that the majority of heparan sulfate proteoglycans and biosynthetic enzymes are proline-rich and core proteoglycan receptor family of Syndecans (Sdc1-4) (FIG. 3D). The data imply that PRS inhibition reduces the translation of the proline-rich heparan sulfate proteoglycans and their biosynthetic enzymes.

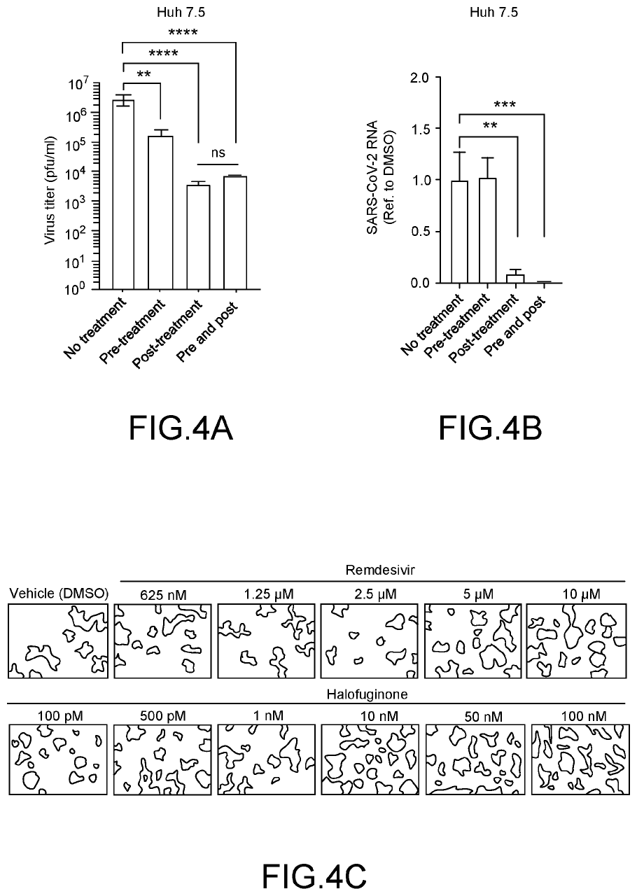

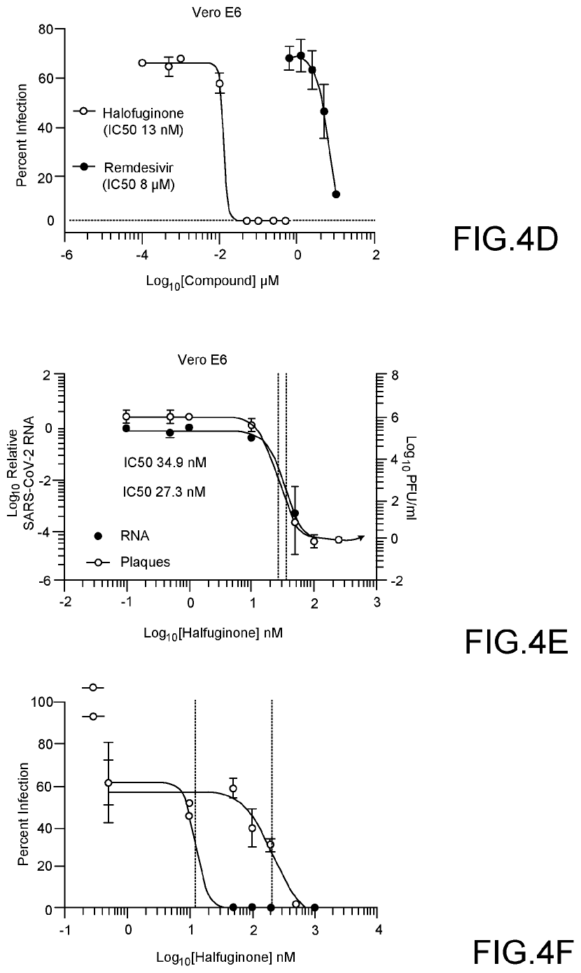

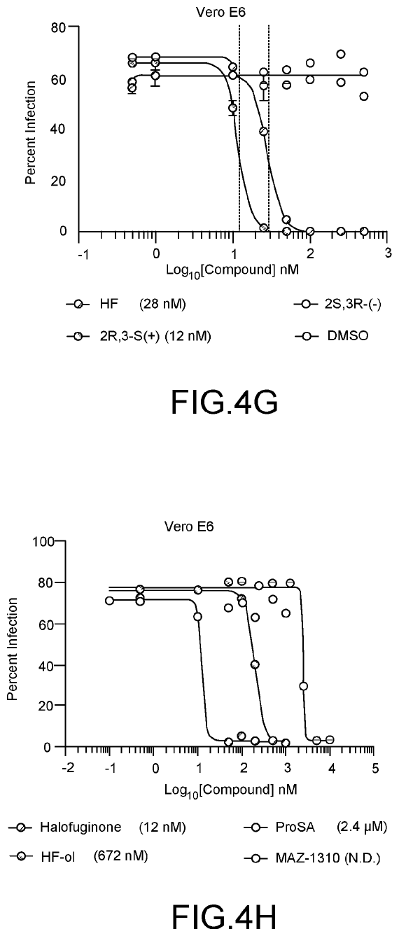

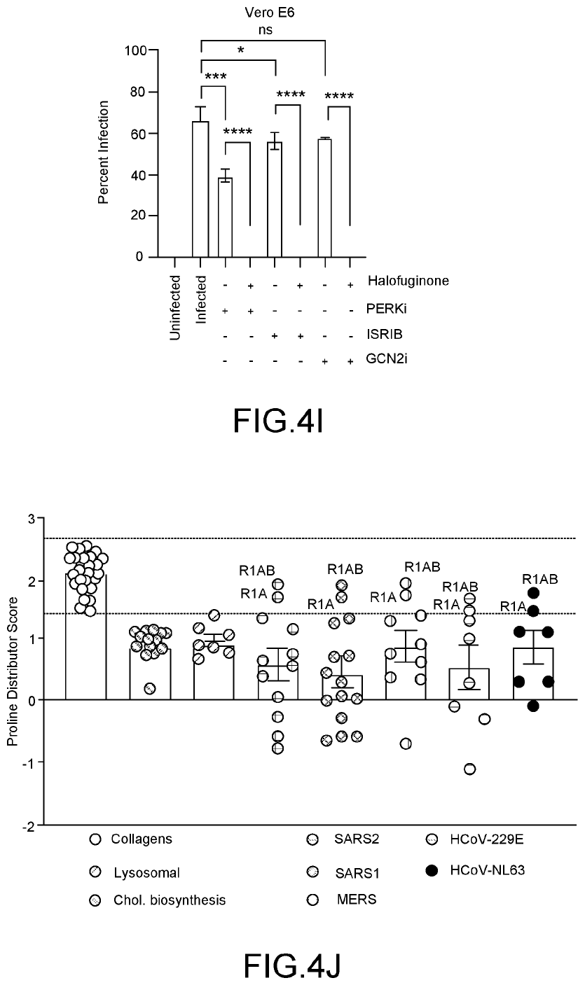

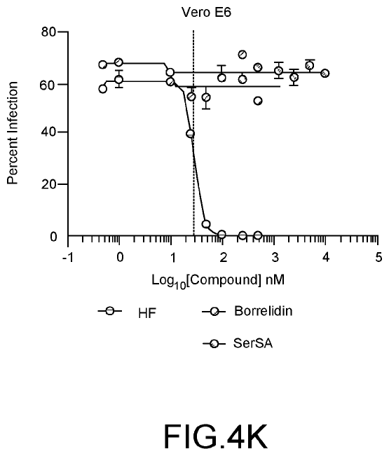

Halofuginone Inhibits Expression and Translation of Heparan Sulfate and Proteoglycans. Halofuginone's effects on the transcriptome were evaluated. Hep3B cells were treated for 6 and 8 hrs in the absence or presence of halofuginone at 220 or 500 nM. RNA-seq analysis revealed halofuginone treatment's dramatic impact on the transcriptome, but with minimal differences between 200 or 500 nM (FIG. 4A-B). Metascape analysis of downregulated genes showed that these belonged to pathways involved in glycoprotein biosynthesis and proteoglycan metabolic processes (FIG. 4C). Indeed, most chondroitin sulfate and heparan sulfate biosynthetic enzymes and core proteoglycans were downregulated at the mRNA level (FIG. 4D). In particular, the heparan sulfate proteoglycan receptors GPC1 and SDC1 were significantly downregulated in conjunction with the biosynthetic enzymes B3GAT3 and EXTL3 (FIG. 4E). No significant difference in expression of host factors exploited by SARS-CoV-2, such as TMPRSS2 and ACE2, were observed. Hence, the data suggest halofuginone inhibits heparan sulfate and proteoglycan production at the translational and transcriptional levels.

Halofuginone Inhibits Live SARS-CoV-2 infection and Replication. Halofuginone mediates a strong inhibition of heparan sulfate mediated attachment of the SARS-CoV-2 Spike protein. However, what matters is the inhibition of live SARS-CoV-2 virion invasion and replication. Initially, Hep3B were challenged, who do not express ACE2 (FIG. 4D), with live SARS-CoV-2 for 4 hrs at MOI 0.1 and pretreated Hep3B cells with halofuginone for 24 hrs pre- and 24 hrs. Post-infection (FIG. 5A). The data show a dramatic inhibition of SARS-C0V-2 infectivity and replication. No virus was detected in Hep3B at halofuginone doses greater than 50 nM (FIG. 5A). Similar results were obtained in HuH 7.5 cells, a cell line widely used to amplify Hepatitis C Virus (HCV) as HCV can replicate in these cells due to a defect in innate antiviral signaling. No virus was detected in plaque assays at doses above 100 nM in HuH 7.5 incubated similarly (FIG. 5A). Given the strong inhibition, whether halofuginone could both inhibit SARS-CoV-2 attachment and invasion as well as replication and secretion of new SARS-CoV-2 virions was explored. To this end, treated HuH 7.5 cells were treated with 100 nM halofuginone, 24 hrs before or after a 4 hr infection with SARS-CoV-2 at MOI 0.1. Both the pretreatment and the post-infection treatment were able to significantly reduce SARS-CoV-2 infectivity by a 3 and 4 Log difference, respectively (FIG. 5B). This observation suggested that halofuginone is also able to inhibit replication and secretion of invaded virions. This inhibition occurs possibly via activation of the integrated stress response, which blocks 5′cap-dependent mRNA translation, which is what coronaviruses hijack to produce new virions in the host. Treatment of HuH 7.5 cells with the integrated stress response inhibitors, inhibited SARS-CoV-2 infectivity by several logs (FIG. 5C) indicated that modulation of the integrated stress response could indeed inhibit viral replication. Vero E6 cells do not present a reduction in spike protein binding and pseudovirus binding (FIGS. 1C & 1G). Hence, if we would see inhibition of SARS-CoV-2 infectivity, this would have to be mediated by inhibiting the replication of SARS-CoV-2. Treating Vero E6 after a 4 hr infection with SARS-CoV-2 at MOI 0.5 with halofuginone resulted in complete inhibition of SARS-CoV-2 infectivity at doses greater than 10 nM (FIG. 5D-E). A direct comparison with Remdesivir (IC50 at 8 μM), a drug currently in the clinic to treat COVID19, revealed halofuginone (IC50 at 13 nM) to be 1000-fold more potent as a SARS-CoV-2 inhibitor (FIG. 5D-E). Finally, treating differentiated primary human upper airway epithelial cells with different halofuginone doses revealed a complete inhibition of SARS-CoV-2 in this human-relevant cell model (FIG. 5F-G).

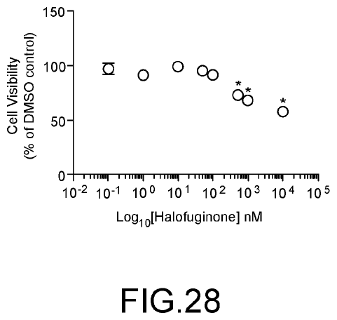

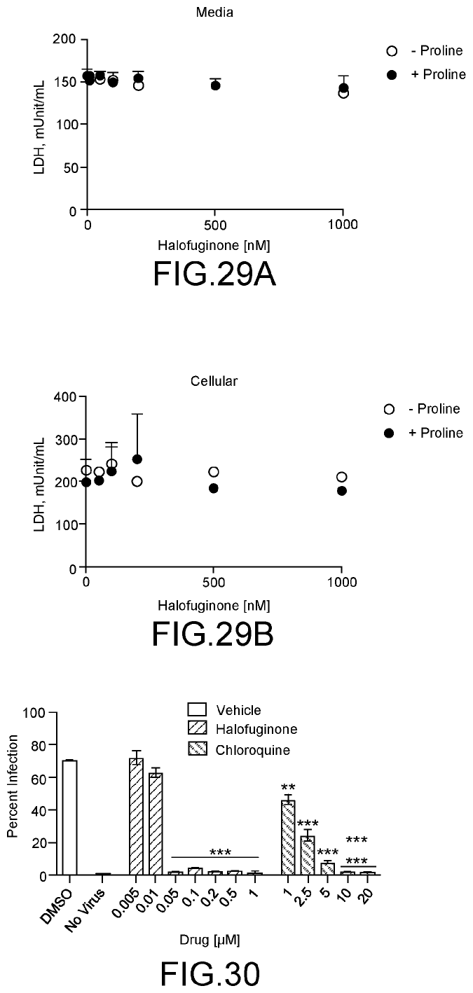

Halofuginone Inhibits Infection and Replication by Authentic SARS-CoV-2. To determine if halofuginone inhibition of SARS-CoV-2 virus production was due to reduced viral entry or subsequent intra-host replication, Huh 7.5 cells were treated with 100 nM halofuginone or vehicle, either before, after, or before and after infection with SARS-CoV-2. Similarly, as in Hep3B halofuginone prevented productive infection of Huh 7.5 without affecting cell viability (FIGS. 4A and B, and FIG. 28). Surprisingly, pretreatment with halofuginone alone significantly reduced SARS-CoV-2 infection by ˜30-fold (FIG. 4A). However, even more impressive is that halofuginone added after viral infection reduced the amount of secreted infectious virions by nearly 1000-fold (FIG. 4A). Intracellular viral RNA did not change when the cells were only treated before infection as expected, but viral RNA levels dramatically decreased 10- to 100-fold when halofuginone was present after infection (FIG. 4B). These observations suggest that halofuginone potently inhibits SARS-CoV-2 viral replication in addition to suppressing HS-dependent infection.

Halofuginone did not decrease cellular HS or the binding of recombinant spike RBD protein in Vero E6 cells. However, given the effects of halofuginone treatment on viral replication, the effect of halofuginone on infection by authentic SARS-CoV-2 in Vero E6 cells was examined as well. Halofuginone completely inhibited SARS-CoV-2 infectivity at 50 nM with an IC₅₀ of 13 nM as measured by immunofluorescent (IF) detection of the nucleocapsid protein and plaque assays (FIGS. 4C-D and 29). In contrast, Remdesivir, which is currently being used experimentally to treat SARS-CoV-2 infections, had a calculated IC₅₀ of 8 μM. Thus, halofuginone showed an unexpected ˜1,000-fold more potent inhibition of infection as compared to Remdesivir in this experimental setup (FIG. 4C-D). Similarly, halofuginone was 100-fold more potent compared to chloroquine (IC₅₀ 1.9 μM) (FIG. 30). Moreover, halofuginone treatment reduced SARS-CoV-2 spike intracellular mRNA levels more than 20,000-fold with an IC₅₀ of 34.9 nM (FIG. 4E).

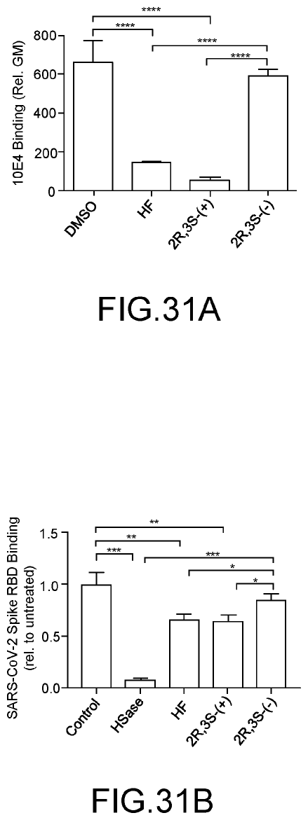

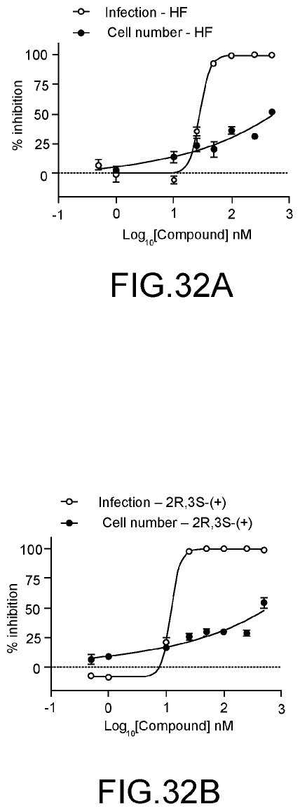

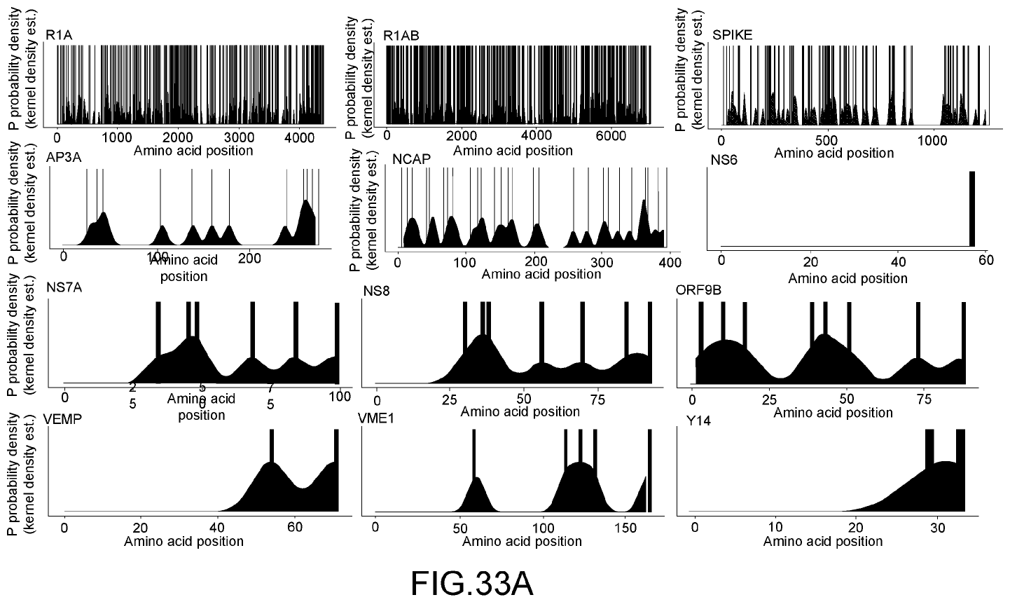

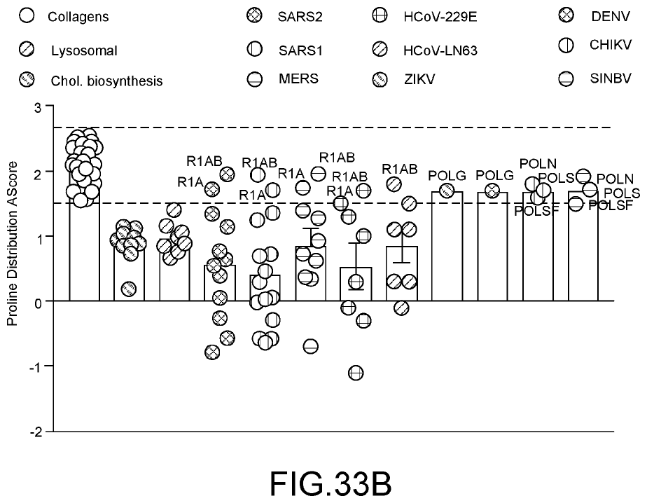

Next, the impact of halofuginone on viral replication was examined to see if halofuginone activity was dependent on PRS inhibition. Halofuginone competes with proline for the PRS active site¹⁵. Addition of excess proline to the media (4 mM) of SARS-CoV-2 infected Vero E6 cells increased the IC₅₀ of halofuginone from 12.5 nM to 210 nM (FIG. 4F). Commercial halofuginone is a racemate of two, 2S,3R-(−) and 2R,3S-(+) enantiomers³⁰. Hence, we evaluated if viral inhibition was due to on-target effects of the PRS targeting 2R,3S-(+) enantiomer (FIG. 4G)³⁰ . The 2R,3S-(+) inhibited SARS-CoV-2 infectivity with an IC₅₀ of 12 nM compared to an IC₅₀ of 28 nM for the racemic mixture. No inhibition was observed when using the non-targeting 2S,3R-(−)enantiomer (FIG. 4G). Only the active 2R,3S-(+) enantiomer inhibited HS production and authentic SARS-CoV-2 spike RBD protein binding (FIGS. 31-32).

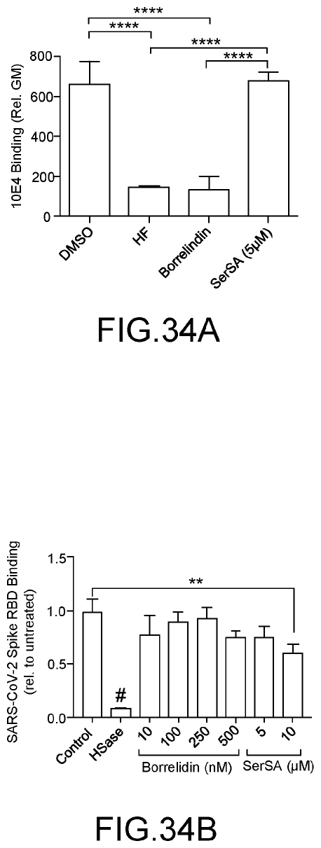

While other PRS inhibitors ProSA and halofuginol inhibited authentic SARS-CoV-2 infection of Vero E6 cells, the effect was suprisingly only observed at significantly higher doses than that of halofuginone (FIG. 4H). The negative control compound, MAZ1310, did not have any activity (FIG. 4H). These results demonstrate that inhibition of PRS activity suppresses SARS-CoV-2 infection (FIG. 4H). Inhibitors of the ISR, as well as inhibitors of eIF2-alpha kinases PERK and GCN2 were unable to attenuate the halofuginone-mediated inhibition of SARS-CoV-2 infectivity (FIG. 4I). Hence, the observed antiviral effect is not dependent on ISR activation, consistent with the effects seen on the binding of recombinant spike RBD protein and mAb 10E4 binding (FIG. 3D-E).^{31.32} Upon cell entry, SARS-CoV-2 genomic RNA is translated into two large polyproteins, pp1a (>400 kDa) and pp1ab (>700 kDa) that undergo proteolytic processing into 11 or 16 non-structural proteins, respectively, many of which are required for RNA replication. Both pp1a (R1A) and pp1ab (R1AB), and to a lesser degree spike protein, have high proline contents comparable to collagens (FIGS. 4J & 33). To probe the importance of the viral protein proline content versus general mRNA translation inhibition, two other AARS inhibitors, borrelidin and seryl-sulfamoyl adenosine (SerSA) that target the threonyl- and seryl-tRNA-synthetase, respectively were evaluated. Neither of these AARS inhibitors were able to significantly attenuate viral replication (FIG. 4K) or Spike protein binding (FIG. 34), suggesting that proline translation is the Achilles heel for SARS-CoV-2 replication and possible other RNA viruses^{33,34}.

Halofuginone is therefore a potent inhibitor of SARS-CoV-2 infection in numerous cell types, including primary human bronchial epithelial cells. This small molecule decreases HS-dependent spike protein binding, SARS-CoV-2 pseudovirus infection, and infection by authentic SARS-CoV-2. Interestingly, it also inhibits authentic SARS-CoV-2 infection post-entry by an HS-independent mechanism. Mechanistically, halofuginone suppresses SARS-CoV-2 infection by inhibiting the PRS, which could suppress the translation of long proline-rich host attachment factors, particularly HSPGs, and SARS-CoV-2 polyproteins pp1a and pp1ab that encode proteins required for viral replication. Thus, halofuginone is a potent host-targeting antiviral with dual inhibitory activity against SARS-CoV-2.

There is a desperate need for a potent, orally bioavailable antiviral for the treatment of COVID-19 that can be administered early in the disease to prevent hospitalization and the development of severe pulmonary disease. The present disclosure provides that halofuginone is a potent inhibitor of SARS-CoV-2 infection with IC₅₀ values in the low nanomolar range in multiple in vitro models of infection. Halofuginone is orally bioavailable and reached an average Cₘₐₓ of 0.54 ng/ml (˜1.3 nM) or 3.09 ng/ml (˜7.4 nM) after a single administration of 0.5 mg or 3.5 mg doses in a phase I clinical trial³⁵. The long half-life (˜3 h) leads to accumulation of halofuginone, with two- to three-fold higher exposure by day 15 of dosing³⁵. Halofuginone widely distributed in tissues after administration in mice with the highest tissue concentrations in the lung and kidney³⁶. Expressed as area under the curve, halofuginone exposure was more than 87-fold higher in the lung compared to plasma after a single intravenous injection in mice³⁶. This suggests that although it may be difficult to obtain halofuginone plasma levels significantly above the IC₅₀ values determined in this disclosure, doses tested in phase I trials can be sufficient to achieve significant anti-SARS-CoV-2 activity in the lung and other organs infected by SARS-CoV-2³⁷⁻⁴⁰.

Excessive inflammation can contribute to inflammatory organ injury during severe COVID-19. There are multiple anti-inflammatory agents under evaluation for the treatment of COVID-19 and low dose dexamethasone recently demonstrated lower 28-day mortality in individuals receiving respiratory supports⁴¹. In addition to the antiviral activity against SARS-CoV-2 that, is described herein, halofuginone has anti-inflammatory and anti-fibrotic activity that can provide yet another additive benefit to individuals with COVID-19 pneumonia^{1,2,15.42-44}.

As shown in the present disclosure, other PRS inhibitors, ProSA and halofuginol, also inhibit infection by authentic SARS-CoV-2 when provided at higher doses. The data suggest that PRS inhibition may be particularly effective at suppressing the production of SARS-CoV-2 polyproteins pp1a and pp1ab that are long and proline-rich. Other positive-sense ssRNA viruses produce long polyproteins that may be similarly sensitive to PRS or other AARS inhibitors. Consistent with this idea, halofuginone demonstrates antiviral activity against future deleterious coronaviruses and other positive ssRNA viruses, including Chikungunya virus and Dengue virus⁴⁵.

In addition to halofuginone, other PRS inhibitors are being evaluated in humans, including DWN12088 that is in phase I clinical trials in Australia and anticipated to be used for the treatment of interstitial pulmonary fibrosis. Thus, evaluation of PRS inhibitors, halofuginone analogs, and other AARS inhibitors could lead to the identification of additional broad-spectrum antiviral agents.

Viruses require host cell resources for replication and thus can be inhibited by therapeutics that target essential host factors. This approach could provide broad activity against diverse viruses while decreasing the risk of emerging viral resistance as the therapy is not directed against a specific virally encoded product. On the other hand, targeting host factors can lead to cytostatic and cytotoxicity. Although inhibition of global translation could lead to excessive toxicity, inhibiting AARSs could provide a more precise way of inhibiting viral proteins and thus limit toxicity. It appears that normal cells are relatively tolerant of decreased AARS levels with minimal effects on global translation. Individuals who are heterozygous carriers for recessive inactivating AARS mutations linked to hypomyelinating leukodystrophy do not display disease phenotypes⁴⁶.

Provided herein is halofuginone as an antiviral agent with potent inhibitory activity against SARS-CoV-2 infection in multiple human cell types. The present disclosure shows that halofuginone reduces both HS and HSPG biosynthesis, which are required for viral adhesion. In addition, it was found that the inhibitory capacity of halofuginone on SARS-CoV-2 infection is not limited to HS reduction, but also shows potent inhibition in the viral replication stage. Mechanistically, the data provided herein suggest that these effects are caused by PRS inhibition and its effect on production of proline-rich proteins and not dependent on ISR activation. This observation is in agreement with previous studies reporting that coronaviruses overcome the inhibitory effects of eIF2α, phosphorylation on viral mRNA translation^{31.32}. Furthermore, many viruses, including coronaviruses (SARS1 and SARS2), shutdown host translation using nonstructural protein I (Nsp1) and activate eIF2α associated PERK dependent stress responses that benefit the virus⁴⁷. Halofuginone can prevent SARS-CoV-2 from circumventing these mechanisms of host protein translational shutdown.

Halofuginone oral administration has been evaluated in a phase I clinical trial in humans and based on pharmacological studies in mice is distributed to SARS-CoV-2 target organs, including the lung.

In conclusion, the present disclosure provides that halofuginone is a potent inhibitor of SARS-CoV-2 infection which emphasizes its potential as an effective treatment for COVID-19 in the clinic. Beyond its antiviral activity, halofuginone has potent anti-inflammatory and anti-fibrotic properties that can provide additive benefit in the treatment of COVID-19 pneumonia. Based on this in vitro preclinical data, halofuginone is an effective antiviral and antifibrotic agent for the treatment of individuals with COVID-19.

### Additional Methods of Use

The disclosure also provides, in various embodiments, a method for treating a subject suffering from a disease or condition, or preventing the subject from contracting the disease or condition. The method comprises administering to the subject a therapeutically effective amount of halofuginone or a pharmaceutically acceptable salt thereof.

In various embodiments, optionally in combination with any other embodiment herein described, a method of treatment of prevention as described herein comprises administering to the subject a prolyl-tRNA-synthetase inhibitors, such as T-3833261 and ProSA.

In other embodiments, optionally in combination with any other embodiment herein described, a method of treatment of prevention as described herein comprises administering to the subject an aminoacyl tRNA-Synthetase inhibitors, such as but not limited to SB-217452.

In other embodiments, optionally in combination with any other embodiment herein described, a method of treatment of prevention as described herein comprises administering to the subject an Integrated Stress Response inhibitor such as, but not limited to, ISRIB, GSK2606414, and GCN2-IN-1.

In other embodiments, optionally in combination with any other embodiment herein described, a method of treatment of prevention as described herein comprises administering to the subject an antibody, siRNA, or ASO targeting the integrated stress response proteins. These therapeutics include but are not limited to ATF4, eIF2alpha and tRNA-synthetases.

### Anemia, Iron Deficiency, and Iron Loading Disorder

In embodiments, the disease or condition is an iron loading disease such as but not limited to iron-refractory iron-deficiency anemia, and anemia of inflammation. In other embodiments, the disease or condition is an iron deficiency disease such as but not limited to, hereditary hemochromatosis and anemia.

Halofuginone inhibits in a dose-dependent fashion the expression of hepcidin, a 25 amino acid peptide hormone encoded by the HAMP gene, a master regulator of iron homeostasis.

Hepcidin is produced and secreted predominantly by hepatocytes and negatively regulates the activity of ferroportin (FPN), the sole cellular iron exporter. FPN mediates the transport of iron from enterocytes, macrophages, and hepatocytes to transferrin (TF) in the circulation 1. Extracellular hepcidin binds FPN and induces its endocytosis and subsequent degradation in the lysosome. Hepcidin can also block FPN iron transport activity directly by blocking its channel. Under normal conditions plasma hepcidin maintains iron homeostasis, preventing excessive iron absorption and iron mobilization from storage tissues.

Aberrant hepcidin expression or reception in humans contributes to a large spectrum of genetic and acquired iron diseases. Hepcidin deficiency or resistance results in iron overload disease. Patients with excessive hepcidin expression present with hypoferremia. The insufficient supply of iron affects erythropoiesis, translating into various forms of anemia, including familial iron-refractory iron-deficiency anemia (IRIDA). Elevated hepcidin levels in IRIDA patients are due to mutations in TMPRSS6. The patients respond poorly to oral iron supplementation and often require intravenous iron therapy, but only partial improvement occurs Anemia of inflammation is one of the two most common anemias worldwide and associated with chronic systemic inflammatory disorders, including rheumatoid arthritis, inflammatory bowel disease, chronic infections, cancer, and systemic inflammation, including chronic liver and chronic kidney disease. Inflammation greatly increases hepcidin synthesis due to IL6 signaling. The preferred treatment option is to reduce the underlying inflammation, but unfortunately, this approach is not always feasible (e.g. in chronic kidney disease). Administration of erythropoietin derivatives is another option, but the overall benefit of this approach remains unclear.

The dearth of treatment options for hepcidin-associated iron-diseases advocates for the development of novel drugs targeting the hepcidin-ferroportin axis.

### Lysosomal Storage Diseases

In some embodiments, the disease or condition is a lysosomal storage disease. A lysosomal storage disease includes but not limited to mucopolysaccharidoses disorders. Halofuginone and derivatives or salts thereof, in accordance with the methods described herein, prevent the production of glycosaminoglycans and their core proteoglycans, the lysosome storage product in the mucopolysaccharidoses disorders.

### Neurodegenerative Disorders

In various embodiments, the disease or condition is a neurodegenerative disorder. A neurodegenerative disorder includes Alzheimer's disease, dementia, Prion disease, stroke, Parkinson's disease, neurodegenerative disorders associated with plaque/amyloid formation including but not limited to, Synuclein, Tau, all of the aggregating protein disorders involve HS and reduction of HS, and Lewy Body disease.

Halofuginone and derivatives as described herein reduce heparan sulfate presentation at the cell surface of neurological cells, which attenuate progression and onset of neurodegenerative disorders. Halofuginone further prevent the production of glycosaminoglycans and their core proteoglycans, which underlies plaque and amyloid formation as well as promoting aggregation of proteins relevant to Lewy Body, Alzheimer, Prion disease and other neuro-aggregation pathologies.

Viruses. In various embodiments, the disease or condition to be treated by the methods disclosed herein is a virus. As illustrated in the examples, the virus is chosen from DENV-2 (Dengue), ZIKV (Zika virus), and HIV-1. Halofuginone exhibits antiviral activity against all these viruses.

### Pharmaceutical Composition

The disclosure provides in another embodiment a pharmaceutical composition comprising a compound described herein or pharmaceutically acceptable salt thereof and a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of a compound or a pharmaceutically acceptable salt thereof that is administered is governed by such considerations, and is the minimum amount necessary to exert a cytotoxic effect on a cancer, or to inhibit protease activity, or both. Such amount may be below the amount that is toxic to normal cells, or the subject as a whole. Generally, the initial therapeutically effective amount of a compound (or a pharmaceutically acceptable salt, stereoisomer, or tautomer thereof) of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of a compound (or a pharmaceutically acceptable salt, stereoisomer, or tautomer thereof) of the present disclosure. In another embodiment, such dosage forms contain from about 50 mg to about 500 mg of a compound (or a pharmaceutically acceptable salt, stereoisomer, or tautomer thereof) of the present disclosure. In yet another embodiment, such dosage forms contain from about 25 mg to about 200 mg of a compound (or a pharmaceutically acceptable salt, stereoisomer, or tautomer thereof) of the present disclosure. In still another embodiment, such dosage forms contain from about 10 mg to about 100 mg of a compound (or a pharmaceutically acceptable salt, stereoisomer, or tautomer thereof) of the present disclosure. In a further embodiment, such dosage forms contain from about 5 mg to about 50 mg of a compound (or a pharmaceutically acceptable salt, stereoisomer, or tautomer thereof) of the present disclosure. In any of the foregoing embodiments the dosage form can be administered once a day or twice per day.

In various embodiments, optionally in combination with any other embodiment described herein, halofuginone is administered as its active diastereomer, 2R,3S-(+) halofuginone. Halofuginone can be administered as pure 2R,3S-(+) halofuginone, or as mixture having a diastereomeric excess (de) of 2R,3S-(+) halofuginone. The amount of 2R,3S-(+) halofuginone can be expressed in terms of percent diastereomeric excess: racemic halofuginone has 0% de, and pure 2R,3S-(+) halofuginone has 100% de. Various percentage de are contemplated, including about 10, 20, 30, 40. 50, 60, 70, 80, 90, 95, 96, 97, 98, and 99.5% de 2R,3S-(+) halofuginone.

Halofuginone as its 2R,3S-(+) diastereomer exhibits at least a two-fold greater anti-viral potency in vivo compared to racemic halofuginone. Accordingly, the therapeutically effective amount of 2R,3S-(+) halofuginone, for use in the methods described herein, is significantly less than the therapeutically effective amount of racemic halofuginone. Thus, in various embodiments, the therapeutically effective amount of 2R,3S-(+) halofuginone is a dose in the range of about 1 μg/kg to about 20 μg/kg. Specific examples include about 1 μg/kg, about 1.5 μg/kg, about 2 μg,/kg, about 2.5 μg/kg, about 3 μg/kg, about 4 μg/kg, about 5 μg/kg, about 10, and about 15 μg/kg. In an illustrative embodiment, the amount of 2R,3S-(+) halofuginone is about 2.5 μg/kg.

In additional embodiments, the therapeutically effective amount of 2R,3S-(+) halofuginone that is administered, such as in a pharmaceutical composition described herein, is an amount that can range from about 0.01 mg to about 0.500 mg. Specific amounts include about 0.050 mg, about 0.100 mg, about 0.125 mg, about 0.150 mg, about 0.175 mg, about 0.200 mg, about 0.225 mg, about 0.250 mg, about 0.275 mg, about 0.300 mg, about 0.325 mg, about 0.350 mg, about 0.375 mg, or about 0.400 mg.

In some embodiments, the therapeutically effective amount of halofuginone or 2R,3S-(+) halofuginone for use in the methods described herein is amount that leads to the inhibition of spike protein binding, such as inhibition in the range of about 10% to about 90%. In other embodiments, the therapeutically effective amount leads to a reduction of cell surface heparan sulfate content, such as a reduction of about 2-fold to about 10-fold.

The compositions of the present disclosure can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

In another aspect, also encompassed are pharmaceutical compositions suitable for single unit dosages that comprise a compound of the disclosure or its pharmaceutically acceptable stereoisomer, salt, or tautomer and a pharmaceutically acceptable carrier.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of the protease inhibitor.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

A compound described herein may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

Numbered references cited in the disclosure above:
1 Pines, M. & Spector, I. Halofuginone—the multifaceted molecule. Molecules 20, 573-594, doi:10.3390/molecules20010573 (2015).
2 Luo, Y., Xie, X., Luo, D., Wang, V. & Gao, Y. The role of halofuginone in fibrosis: more to be explored? J Leukoc Biol 102, 1333-1345, doi:10.1189/jlb.3RU0417-148RR (2017).
3 Clausen, T. M. et al. SARS-CoV-2 Infection Depends on Cellular Heparan Sulfate and ACE2. Cell, doi:https://doi.org/10.1016/j.cell.2020.09.033 (2020).
4 Beigel, J. H. et al. Remdesivir for the Treatment of Covid-19—Preliminary Report. N Engl J Med, doi:10.1056/NEJMoa2007764 (2020).
5 Johns Hopkins University and Medicine, Johns Hopkins Coronavirus Resource Centre. https://coronavirus.jhu.edu (2020).
6 Food and Drug Administration. https://www.fda.gov/news-events/press-announcements/coronavirus-covid-19-update-fda-issues-einergency-use-authorization-potential-covid-19-treatment (2020).
7 Gillenwater, S., Rahaghi, F. & Hadeh, A. Remdesivir for the Treatment of Covid-19—Preliminary Report. N Engl J Med 383, doi:10.1056/NEJMc2022236 (2020).
8 McMahon, J. H., Udy, A. & Peleg, A. Y. Remdesivir for the Treatment of Covid-19—Preliminary Report. N Engl J Med 383, doi:10.1056/NEJMc2022236 (2020).
9 Wang, Y. et al. Remdesivir in adults with severe COVID-19: a randomised, double-blind, placebo-controlled, multicentre trial. Lancet 395, 1569-1578, doi:10.1016/S0140-6736(20)31022-9 (2020).
10 Spinner, C. D. et al. Effect of Remdesivir vs Standard Care on Clinical Status at 11 Days in Patients With Moderate COVID-19: A Randomized Clinical Trial. JAMA, doi:10.1001/jama.2020.16349 (2020).
11 McCreary, E. K. & Angus, D. C. Efficacy of Remdesivir in COVID-19. JAMA, doi:10.1001/jama.2020.16337 (2020).
12 Thorp, H. H. A dangerous rush for vaccines. Science 369, 885, doi:10.1126/science.abe3147 (2020).
13 Mycroft-West, C. J. et al. Heparin inhibits cellular invasion by SARS-CoV-2: structural dependence of the interaction of the surface protein (spike) S1 receptor binding domain with heparin. bioRxiv, 2020.2004.2028.066761, doi:10.1101/2020.04.28.066761 (2020).
14 Hoffmann, M. et al. SARS-CoV-2 Cell Entry Depends on ACE2 and TMPRSS2 and Is Blocked by a Clinically Proven Protease Inhibitor. Cell 181, 271-280 e278, doi:10.1016/j.cell.2020.02.052 (2020).
15 Keller, T. L. et al. Halofuginone and other febrifugine derivatives inhibit prolyl-tRNA synthetase. Nat Chem Biol 8, 311-317, doi:10.1038/nchembio 790 (2012).
16 Cribbs, A. et al. Inhibition of histone H3K27 demethylases selectively modulates inflammatory phenotypes of natural killer cells. J Biol Chem 293, 2422-2437, doi:10.1074/jbc.RA117.000698 (2018).
17 Cribbs, A. P. et al. Histone H3K27me3 demethylases regulate human Th17 cell development and effector functions by impacting on metabolism. Proc Natl Acad Sci USA 117, 6056-6066, doi:10.1073/pnas.1919893117 (2020).
18 Cottone, L. et al. Inhibition of histone H3K27 demethylases inactivates brachyury (TBXT) and promotes chordoma cell death. Cancer Res, doi:10.1158/0008-5472.CAN-20-1387 (2020).
19 Zhou, H., Sun, L., Yang, X. L. & Schimmel, P. ATP-directed capture of bioactive herbal-based medicine on human tRNA synthetase. Nature 494, 121-124, doi:10.1038/nature11774 (2013).
20 Blight, K. J., McKeating, J. A. & Rice, C. M. Highly permissive cell lines for subgenomic and genomic hepatitis C virus RNA replication. J Virol 76, 13001-13014, doi:10.1128/jvi.76.24.13001-13014.2002 (2002).
21 Pessentheiner, A. R., Ducasa, G. M. & Gordts, P. Proteoglycans in Obesity-Associated Metabolic Dysfunction and Meta-Inflammation. Front Immunol 11, 769, doi:10.3389/fimmu.2020.00769 (2020).
22 David, G., Bai, X. M., Van der Schueren, B., Cassiman, J. J. & Van den Berghe, H. Developmental changes in heparan sulfate expression. in situ detection with mAbs. J Cell Biol 119, 961-975, doi:10.1083,jcb.119.4.961 (1992).
23 Zhou, Y. et al. Metascape provides a biologist-oriented resource for the analysis of systems-level datasets. Nat Commun 10, 1523, doi:10.1038/s41467-019-09234-6 (2019).
24 Francklyn, C. S. & Mullen, P. Progress and challenges in aminoacyl-tRNA synthetase-based therapeutics. J Biol Chem 294, 5365-5385, doi:10.1074/jbc.REV118.002956 (2019).
25 Adachi, R. et al. Discovery of a novel prolyl-tRNA synthetase inhibitor and elucidation of its binding mode to the ATP site in complex with 1-proline. Biochem Biophys Res Commun 488, 393-399, doi:10.1016/j.bbrc.2017.05.064 (2017).
26 Costa-Mattioli, M. & Walter, P. The integrated stress response: From mechanism to disease. Science 368, doi:10.1126/science.aat5314 (2020).
27 Fusakio, M. E. et al. Transcription factor ATF4 directs basal and stress-induced gene expression in the unfolded protein response and cholesterol metabolism in the liver. Mol Biol Cell 27, 1536-1551, doi:10.1091/mbc.E16-01-0039 (2016).
28 Kamtekar, S. et al. The structural basis of cysteine aminoacylation of tRNAPro by prolyl-tRNA synthetases. Proc Natl Acad Sci USA 100, 1673-1678, doi:10.1073/pnas.0437911100 (2003).
29 Nakamura, A. et al. Inhibition of GCN2 sensitizes ASNS-low cancer cells to asparaginase by disrupting the amino acid response. Proc Natl Acad Sci USA 115, E7776-E7785, doi:10.1073/pnas.1805523115 (2018).
30 Jain, V. el al. Structure of Prolyl-tRNA Synthetase-Halofuginone Complex Provides Basis for Development of Drugs against Malaria and Toxoplasmosis. Structure 23, 819-829, doi:10.1016/j.str.2015.02.011 (2015).
31 Krahling, V., Stein, D. A., Spiegel, M., Weber, F. & Muhlberger, E. Severe acute respiratory syndrome coronavirus triggers apoptosis via protein kinase R but is resistant to its antiviral activity. J Virol 83, 2298-2309, doi:10.1128/JVI.01245-08 (2009).
32 Nakagawa, K., Lokugamage, K. G. & Makino, S. Viral and Cellular mRNA Translation in Coronavirus-Infected Cells. Adv Virus Res 96, 165-192, doi:10.1016/bs.aivir.2016.08.001 (2016).
33 Huter, P. et al. Structural Basis for Polyproline-Mediated Ribosome Stalling and Rescue by the Translation Elongation Factor EF-P. Mol Cell 68, 515-527 e516, doi:10.1016/j.molcel.2017.10.014 (2017).
34 Pavlov, M. Y. et al. Slow peptide bond formation by proline and other N-alkylamino acids in translation. Proc Acad Sci USA 106, 50-54, doi:10.1073/pnas.0809211106 (2009).
35 de Jonge, M. J. el al. Phase I and pharmacokinetic study of halofuginone, an oral quinazolinone derivative in patients with advanced solid tumours. Eur J Cancer 42, 1768-1774, doi:10.1016/j.ejca.2005.12.027 (2006).
36 Stecklair, K. P. et al. Pharmacokinetics and tissue distribution of halofuginone (NSC 713205) in CD2F1 mice and Fischer 344 rats. Cancer Chemother Pharmacol 48, 375-382, doi:10.1007/s002800100367 (2001).
37 Ackermann, M. et al. Pulmonary Vascular Endothelialitis, Thrombosis, and Angiogenesis in Covid-19. N Engl J Med 383, 120-428, doi:10.1056/NEJMoa2015432 (2020).
38 Chan, J. F. et al. Simulation of the clinical and pathological manifestations of Coronavirus Disease 2019 (COVID-19) in golden Syrian hamster model. implications for disease pathogenesis and transmissibility. Clin Infect Dis, doi:10.1093/cid/ciaa325 (2020).
39 Gupta, A. et al. Extrapulmonary manifestations of COVID-19. Nat Med 26, 1017-1032, doi:10.1038/s41591-020-0968-3 (2020).
40 Puelles, V. G. et al. Multiorgan and Renal Tropism of SARS-CoV-2. N Engl J Med 383, 590-592, doi:10.1056/NEJMc2011400 (2020).
41 Group, R. C. et al. Dexamethasone in Hospitalized Patients with Covid-19—Preliminary Report. N Engl J Med, doi:10.1056/NEJMoa.2021436 (2020).
42 Battu, S. et al. Amino acid starvation sensing dampens IL-1beta production by activating riboclustering and autophagy. PLoS Biol 16, e2005317, doi:10.1371/journal.pbio.2005317 (2018).
43 Luo, L. et al. Halofuginone attenuates intervertebral discs degeneration by suppressing collagen I production and inactivating TGFbeta and NF-small ka, CyrillicB pathway. Biomed Pharmacother 101, 745-753, doi:10.1016,j.biopha.2018.01.100 (2018).
44 Sundrud, M. S. et al. Halofuginone inhibits TH17 cell differentiation by activating the amino acid starvation response. Science 324, 1334-1338, doi:10.1126/science.1172638 (2009).
45 Hwang, J., Jiang, A. & Fikrig, E. A potent prolyl tRNA synthetase inhibitor antagonizes Chikungunya and Dengue viruses. Antiviral Res 161, 163-168, doi:10.1016/j.antiviral.2018.11.017 (2019).
46 Kwon, N. H, Fox, P. L. & Kim, S. Aminoacyl-tRNA synthetases as therapeutic targets. Nat Rev Drug Discov 18, 629-650, doi:10.1038/s41573-019-0026-3 (2019).
47 Thorns, M. et al. Structural basis for translational shutdown and immune evasion by the Nsp1 protein of SARS-CoV-2. Science, doi:10.1126/science.abc8665 (2020).
48 Son, J. et al. Conformational changes in human prolyl-tRNA synthetase upon binding of the substrates proline and ATP and the inhibitor halofuginone. Acta Crystallogr D Biol Crystallogr 69, 2136-2145, doi:10.1107/S0907444913020556 (2013).
49 Song, Y. et al. Double mimicry evades tRNA synthetase editing by toxic vegetable-sourced non-proteinogenic amino acid. Nat Commun 8, 2281, doi:10.1038/s41467-017-02201-z (2017).

### Experimental Protocols

SARS-CoV-2 spike protein production. Recombinant SARS-CoV-2 spike protein, encoding residues 1-1138 (Wuhan-Hu-1; GenBank: MN908947.3) with proline substitutions at amino acids positions 986 and 987 and a “GSAS” substitution at the furin cleavage site (amino acids 682-682), was produced in ExpiCHO cells by transfection of 6×10⁶ cells/ml at 37° C. with 0.8 μg/ml of plasmid DNA using the ExpiCHO expression system transfection kit in ExpiCHO Expression Medium (ThermoFisher). One day later the cells were refed, then incubated at 32° C. for 11 days. The conditioned medium was mixed with cOmplete EDTA-free Protease Inhibitor (Roche). Recombinant protein was purified by chromatography on a Ni²⁺ Sepharose 6 Fast Flow column (1 ml, GE LifeSciences). Samples were loaded in ExpiCHO Expression Medium supplemented with 30 mM imidazole, washed in a 20 mM Tris-Cl buffer (pH 7.4) containing 30 mM imidazole and 0.5 M NaCl. Recombinant protein was eluted with buffer containing 0.5 M NaCl and 0.3 M imidazole. The protein was further purified by size exclusion chromatography (HiLoad 16/60 Superdex 200, prep grade. GE LifeSciences) in 20 mM HEPES buffer (pH 7.4) containing 0.2 M NaCl. Recombinant ectodomains migrate as a trimer assuming a monomer molecular mass of ˜142,000 and 22 N-linked glycans per monomer. SDS-PAGE, TEM analysis, and SEC studies validate protein purity and the presence of trimers.

Biotinylation. For binding studies, recombinant spike protein and ACE2 was conjugated with EZ-Link™ Sulfo-NHS-Biotin (1:3 molar ratio; Thermo Fisher) in Dulbecco's PBS at room temperature for 30 min. Glycine (0.1 M) was added to quench the reaction and the buffer was exchanged for PBS using a Zeba spin column (Thermo Fisher).

Reagents. SARS-CoV-2 (2019-nCoV) Spike Protein (RBD, His Tag) (Sino Biologicals, 40592-V08B), and proteins were biotinylated using EZ-Link™ Sulfa-NHS-Biotin, No-Weigh™ (Thermo Fisher Scientific, A39256). Heparin lyases were from IBEX pharmaceuticals, Heparinase I (IBEX, 60-012), Heparinase II (IBEX, 60-018), Heparinase III (IBEX, 60-020). Protein production reagents included, Pierce™ Protein Concentrators PES Thermo Scientific™, Pierce™ Protein Concentrator PES (Thermo Fisher Scientific, 88517) and Zeba™ Spin Desalting Columns, 40K MWCO, 0.5 mL (Thermo Fisher Scientific, 87766). ISR pathway inhibitors used are ISRIB (Sigma, SML0843), GSK2606414 (Sigma, 516535), and A-92 (Axon, 2720). Antibodies used were, anti-Spike antibody [1A9] (GeneTex, GTX632604), anti-Nucleocapsid antibody (GeneTex, GTX135357), Anti-HS (Clone F58-10E4) (Fisher Scientific, NC1183789), and 3G10 (BioLegend, 4355S). Secondary antibodies were, Anti-His-HRP (Genscript, A00612), Avidin-HRP (Biolegend, 339902), and Streptavadin-Cy5 (Thermo Fisher, SA1011). Luciferase activity was monitored by Bright-Glo™ (Promega, E2610). All cell culture medias and PBS where from Gibco.

Cell Culture. Huh-7.5 cells¹ were generously provided by Charles M. Rice (Rockefeller University, New York, NY). Hep3B, BHK-15 and Calu-3 cells were from ATCC and were grown in DMEM medium containing 10% % fetal bovine serum, and 100 IU/ml of penicillin, 100 μg/ml of streptomycin sulfate and nonessential amino acids. BHK-15 were grown in Modified Eagle's supplemented with 10% fetal bovine serum and nonessential amino acids (Gibco, #11140-050). Hep3B, A375 and Vero E6 cells were from ATCC. The Hep3B cells carrying mutations in HS biosynthetic enzymes was derived from the parent ATCC Hep3B stock, and have been described previously². All cells were supplemented with 10% FBS, 100 IU/ml of penicillin and 100 μg/ml of streptomycin sulfate and grown under an atmosphere of 5% CO2 and 95% air. Cells were passaged before 80% confluence was reached and seeded as explained for the individual assays. Cell viability was measured using LDH leakage (Promega) or alamarBleu (Thermo Fisher).

SARS-CoV-2 spike RBD protein production. Recombinant SARS-CoV-2 RBD (GenBank: MN975262.1; amino acid residues 319-529) was cloned into pVRC vector containing a HRV 3C-cleavable C-terminal SBP-His_{8X} tag was produced in ExpiCHO cells by transfection of 6×10⁶ cells/ml at 37° C. with 0.8 μg/ml of plasmid DNA using the ExpiCHO expression system transfection kit in ExpiCHO Expression Medium (ThermoFisher). One day later the cells were fed with ExpiCho Feed, treated with ExpCho Enhancer, and then incubated at 32° C. for 11 days. The conditioned medium was mixed with cOmplete EDTA-free Protease Inhibitor (Roche). Recombinant protein was purified by chromatography on a Ni²⁺ Sepharose 6 Fast Flow column (1 ml, GE LifeSciences). Samples were loaded in ExpiCHO Expression Medium supplemented with 30 mM imidazole, washed in a 20 mM Tris-Cl buffer (pH 7.4) containing 30 mM imidazole and 0.5 M NaCl. Recombinant protein was eluted with buffer containing 0.5 M NaCl and 0.3 M imidazole. The protein was further purified by size exclusion chromatography (HiLoad 16/60 Superdex 200, prep grade. GE LifeSciences) in 20 mM HEPES buffer (pH 7.4) containing 0.2 M NaCl.

Flow cytometry. Cells at 50-80% confluence were lifted with PBS containing 10 mM EDTA (Gibco) and washed in PBS containing 0.5% BSA. The cells were seeded into a 96-well plate at 10⁵ cells per well. In some experiments, a portion of the cells was treated with HSase mix (2.5 mU/ml HSase 1, 2.5 mU/ml HSase II, and 5 mU/ml HSase III; IBEX) for 30 min at 37° C. in PBS containing 0.5% BSA. They were incubated for 30 min at 4° C. with biotinylated spike protein (S1/S2 or RBD; 20 μg/ml or serial dilutions) in PBS containing 0.5% BSA. The cells were washed twice and then reacted for 30 min at 4° C. with Streptavadin-Cy5 (Thermo Fisher; 1:1000 dilution) in PBS containing 0.5% BSA. The cells were washed twice and then analyzed using a FACSCalibur or a FACSCanto instrument (BD Bioscience). All experiments were done a minimum of three separate times in three technical replicates. Data analysis was performed using FlowJo software and statistical analyses were done in Prism 8 (Graph Pad).

qPCR. mRNA was extracted from the cells using TRIzol (Invitrogen) and chloroform and purified using the RNeasy Kit (Qiagen). cDNA was synthesized from the mRNA using random primers and the SuperScript III First-Strand Synthesis System (Invitrogen). SYBR Green Master Mix (Applied Biosystems) was used for qPCR following the manufacturer's instructions, and the expression of TBP was used to normalize the expression of ACE2 between the samples. The qPCR primers used were as follows: ACE2 (human) forward: 5′-CGAAGCCGAAGACCTGTTCTA-3′ and reverse: 5′-GGGCAAGTGTGGACTGTTCC-3′; and TBP (human) forward: 5′-AACTTCGCTTCCGCTGGCCC-3′ and reverse: 5′-GAGGGGAGGCCAAGCCCTGA-3′.

Preparation and infection by pseudotyped VSV. Vesicular Stomatitis Virus (VSV) pseudotyped with spike proteins of SARS-CoV-2 were generated according to a published protocol. Briefly, HEK293T, transfected to express full length SARS-CoV-2 spike proteins, were inoculated with VSV-G pseudotyped ΔG-luciferase or GFP VSV (Kerafast, MA). After 2 hr at 37° C., the inoculum was removed and cells were refed with DMEM supplemented with 10% FBS, 50 U/mL penicillin, 50 μg/mL streptomycin, and VSV-G antibody (II, mouse hybridoma supernatant from CRL-2700, ATCC). Pseudotyped particles were collected 20 hr post-inoculation, centrifuged at 1,320×g to remove cell debris and stored at −80° C. until use.

Cells were seeded at 10,000 cells per well in a 96-well plate. The cells (60-70% confluence) were treated with HSases for 30 min at 37° C. in serum-free DMEM. Culture supernatant containing pseudovirus (20-100 μL) was adjusted to a total volume of 100 μL with PBS, HSase mix or the indicated inhibitors and the solution was added to the cells. After 4 hr at 37° C. the media was changed to complete DMEM. The cells were then incubated for 16 hr to allow expression of reporter gene. Cells infected with GFP containing virus were visualized by fluorescence microscopy and counted by flow cytometry. Cells infected with Luciferase containing virus were analyzed by Bright-Glo™ (Promega) using the manufacturers protocol. Briefly, 100 μL of luciferin lysis solution was added to the cells and incubated for 5 min at room temperature. The solution was transferred to a black 96-well plate and luminescence was detected using an EnSpire multimodal plate reader (Perkin Elmer). Data analysis and statistical analysis was performed in Prism 8.

Virus plaque assays. Confluent monolayers of Vero E6 or Hep3B cells were infected with SARS-CoV-2 at an MOI of 0.1. After one hour of incubation at 37° C., the virus was removed, and the medium was replaced. After 48 hr, cell culture supernatants were collected and stored at −80° C. Virus titers were determined by plaque assays on Vero E6 monolayers. In short, serial dilutions of virus stocks in Minimum Essential Media MEM medium (Gibco, #41500-018) supplemented with 2% FBS was added to Vero E6 monolayers on 24-well plates (Greiner bio-one, #662160) and rocked for 1 hr at room temperature. The cells were subsequently overlaid with MEM containing 1% cellulose (Millipore Sigma, #435244), 2% FBS, and 10 mM HEPES buffer, pH 7.5 (Sigma #H0887) and the plates were incubated at 37° C. under an atmosphere of 5% CO₂/95% air for 48 hr. The plaques were visualized by fixation of the cells with a mixture of 10% formaldehyde and 2% methanol (v/v in water) for 2 hr. The monolayer was washed once with PBS and stained with 0.1% Crystal Violet (Millipore Sigma #V5265) prepared in 20% ethanol. After 15 min, the wells were washed with PBS, and plaques were counted to determine the virus titers. All work with the SARS-CoV-2 was conducted in Biosafety Level-3 conditions either at the University of California San Diego or at the Eva J Pell Laboratory, The Pennsylvania State University, following the guidelines approved by the Institutional Biosafety Committees.

HS and CS digestion and MS analysis. For HS quantification and disaccharide analysis, purified GAGs were digested with a mixture of heparin lyases I-III (2 mU each) for 2 hr at 37° C. in lyase buffer (40 mM ammonium acetate and 3.3 mM calcium acetate, pH 7.0). For CS quantification and disaccharide analysis, the GAGs were digested with 20 mU/mL Chondroitinase ABC (from Proteus vulgaris, Sigma Aldrich) and incubated for 2 hr at 37° C. in lyase buffer (50 mM Tris and 50 mM NaCl, pH 8.0). The reactions were dried in a centrifugal evaporator and tagged by reductive amination with [¹²C₆]aniline. The HS and CS samples were analyzed by liquid chromatography (LC) coupled to tandem mass spectrometry (MS/MS) and quantified by inclusion of [¹³C₆]aniline-tagged standard HS disaccharides (Sigma-Aldrich), as described (Lawrence et al., 2008). The samples were separated on a reverse phase column (TARGA C18, 150 mm×1.0 mm diameter, 5 μm beads, Higgins Analytical, Inc.) using 5 mM dibutylamine as an ion pairing agent (Sigma-Aldrich), and ions were monitored in negative mode. Separation was performed using the same gradient, capillary temperature, and spray voltage as described (Lawrence et al., 2008). The analysis was done on an LTQ Orbitrap Discovery electrospray ionization mass spectrometer (Thermo Scientific) equipped with an Ultimate 3000 quaternary HPLC pump (Dionex).

Western Blot Analysis. Cells were lysed using RIPA buffer, and protein was quantified using a BCA assay. Protein was analyzed by SDS-PAGE on 4-12% Bis-Tris gradient gels (NuPage; Invitrogen) with an equal amount of protein loading. Proteins were visualized after transfer to Immobilon-FL PVDF membrane (Millipore). Membranes were blocked with Odyssey blocking buffer (LI-COR Biosciences) for 30 min and incubated overnight at 4° C. with 3G10 antibodies. Mouse antibodies were incubated with secondary Odyssey IR dye antibodies (1:14,000) and visualized with an Odyssey IR imaging system (LI-COR Biosciences).

RNA-seq library preparation. Cells were lysed in Trizol and total RNA was extracted using the Direct-zol kit (Zymo Research, CA USA). On column DNA digestion was also performed with DNAse treatment. Poly(A) RNA was selected using the NEBNext Poly(A) mRNA Magnetic Isolation module (New England Biolabs) and libraries were prepared using the NEBNext Ultra Directional RNA Library Prep Kit (New England Biolabs) and sequenced using a NextSeq 500 (Illumina). Samples were sequenced at a minimum depth of 15 million reads per sample, paired end with a read length of 2×41 bp.

RNA sequencing data analysis. A computational pipeline was written calling scripts from the CGAT toolkit to analyse the RNA sequencing data (https://github.com/cgat-developers/cgat-flow)^{4.5}. Briefly, FASTQ files were generated and assessed for quality using FASTQC, aligned to GRCh38 (hg-38) and then aligned to the transcriptome using hisat2 v2.1.0⁶. To count mapped reads to individual genes, feature counts v1.4.6, part of the subreads package⁷, was used. Differential gene expression analysis was performed using DESeq2 using treatment and time as factors in the model. Genes were considered to be differentially expressed based on log 2 fold change and p-value <0.05. R scripts used to analyze the transcriptomic data are available through GitHub (https://github.com/Acribbs/deseq2_report). Motif enrichment was performed using homer as described before^{8,9}.

Preparation and infection by pseudotyped VSV. Vesicular Stomatitis Virus (VSV) pseudotyped with spike proteins of SARS-CoV-2 were generated according to a published protocol¹⁰. Briefly, HEK293T, transfected to express full length SARS-CoV-2 spike proteins, were inoculated with VSV-G pseudotyped ΔG-luciferase or GFP VSV (Kerafast, MA). After 2 hr at 37° C., the inoculum was removed and cells were refed with DMEM supplemented with 10% FBS, 50 U/mL penicillin, 50 μg/mL streptomycin, and VSV-G antibody (I1, mouse hybridoma supernatant from CRL-2700; ATCC). Pseudotyped particles were collected 20 hr post-inoculation, centrifuged at 1,320×g to remove cell debris and stored at −80° C. until use.

Cells were seeded at 10,000 cells per well in a 96-well plate. The cells were then treated with Halufuginone for 16 hrs. As a control for HS dependent infection some cells were treated with HSases for 30 min at 37° C. in serum-free DMEM. Culture supernatant containing pseudovirus (20-100 μL) was adjusted to a total volume of 100 μL with PBS, HSase mix or the indicated inhibitors and the solution was added to the cells. After 4 hr at 37° C. the media was changed to complete DMEM. The cells were then incubated for 16 hr to allow expression of the luciferase gene. Cells were analyzed for infection by Bright-Glo™ (Promega) using the manufacturers protocol. Briefly, 100 μL of luciferin lysis solution was added to the cells and incubated for 5 min at room temperature. The solution was transferred to a black 96-well plate and luminescence was detected using an EnSpire multimodal plate reader (Perkin Elmer). Data analysis and statistical analysis was performed in Prism 8.

SARS-CoV-2 infection. SARS-CoV-2 isolate USA-WA1/2020 (BEI Resources) was propagated and infectious units quantified by plaque assay using Vero E6 (ATCC) cells. Approximately 10e4 Vero E6 cells per well were seeded in a 96 well plate and incubated overnight. The following day, cells were washed with PBS and 100 uL of SARS-CoV-2 (MOI 0.5) diluted in serum free DMEM was added per well and incubated 1 h at 37° C. with rocking every 10-15 min. After 1 h, virus was removed, cells washed with PBS and compounds or controls were added at the indicated concentrations. In experiments using inhibitors of the ISR or its related kinases, these compounds were added after viral infection and incubated for 1 h prior to the addition of HF. For viral RNA quantification, cells were washed twice with PBS and lysed in 200 ul TRIzol (ThermoFisher). For immunofluorescence, cells were washed twice with PBS and incubated in 4% formaldehyde for 30 minutes at room temperature. For plaque assays, supernatant was removed and stored at −80 until plaque assays were performed.

### Methods for Human Bronchial Epithelial Cell ALI Generation and Infection

Air Liquid interface. Human Bronchial Epithelial Cells (HBECs, Lonza) were cultured in T75 flasks in PneumaCult-Ex Plus Medium according to manufacturer instructions (StemCell Technologies). To generate air-liquid interface cultures, HBECs were plated on collagen I-coated 24 well transwell inserts with a 0.4-micron pore size (Costar, Corning) at 5×10⁴ cells/ml. Cells were maintained for 3-4 days in PneumaCult-Ex Plus Medium until confluence, then changed to PneumaCult-ALI Medium (StemCell Technologies) containing ROCK inhibitor (Y-27632, Tocris) for 4 days. Fresh medium, 100 μl in the apical chamber and 500 μl in the basal chamber, was added daily. At day 7, the medium in the apical chambers was removed, and the basal chambers were changed every 2-3 days with apical washes with PBS every week for 28 days.

Human bronchial epithelial cell ALI Infection. The apical side of the HBEC ALI culture was gently washed three times with 200 μl of phosphate buffered saline without divalent cations (PBS−/−). An MOI of 0.5 of SARS-CoV-2 live virus in 100 μl total volume of PBS was added to the apical chamber with either DMSO, Heparinase or or various halofuginone concentrations. Cells were incubated at 37° C. and 5% CO2 for 4 hours. Unbound virus was removed, the apical surface was washed and the compounds were re-added to the apical chamber. Cells were incubated for another 20 hours at 37° C. and 5% CO2. After inoculation, cells were washed once with PBS−/− and 100 μl TrypLE (ThermoFisher) was added to the apical chamber then incubated for 10 min in the incubator. Cells were gently pipetted up and down and transferred into a sterile 15 ml conical tube containing neutralizing medium of DMEM+3% FBS. TrypLE was added again for 3 rounds of 10 minutes for a total of 30 min to clear transwell membrane. Cells were spun down and resuspended in PBS with Zombie UV viability dye for 15 min in room temp. Cells were washed once with FACS buffer then fixed in 4% PFA for 30 min at room temp. PFA was washed off and cells were resuspended in PBS.

Virus plaque assays. Confluent monolayers of Vero E6 or Hep3B cells were infected with SARS-CoV-2 at an MOI of 0.1. After one hour of incubation at 37° C., the virus was removed, and the medium was replaced. After 48 hr, cell culture supernatants were collected and stored at −80° C. Virus titers were determined by plaque assays on Vero E6 monolayers. In short, serial dilutions of virus stocks in Dulbecco's Modified Essential Media (DMEM) (Corning, #10-014-CV) were added to Vero E6 monolayers on 12-well plates and incubated 1 hr at 37° C. with rocking every 10-15 min. The cells were subsequently overlaid with MEM containing 0.6% agarose (ThermoFisher Scientific, #16500-100), 4% FBS, non-essential amino acids, L glutamine, and sodium bicarbonate and the plates were incubated at 37° C. under an atmosphere of 5% CO₂/95% air for 48 hr. The plates were fixed with a mixture of 10% formaldehyde and 2% methanol (v/v in PBS) for 24 hr. Agarose overlays were removed, and the monolayer was washed once with PBS and stained with 0.025% Crystal Violet prepared in 2% ethanol. After 15 min, Crystal Violet was removed, and plaques were counted to determine the virus titers. Plaque assays were performed and counted by a blinded experimenter. All work with SARS-CoV-2 was conducted in Biosafety Level-3 conditions either at the University of California San Diego or at the Eva J Pell Laboratory, The Pennsylvania State University, following the guidelines approved by the Institutional Biosafety Committees.

RNA extraction, cDNA synthesis and qPCR. RNA was purified from TRIzol lysates using Direct-zol RNA Microprep kits (Zymo Research) according to manufacturer recommendations that included DNase treatment. RNA was converted to cDNA using the iScript cDNA synthesis kit (BioRad) and qPCR was performed using iTaq universal SYBR green supermix (BioRad) and an ABI 7300 real-time per system. cDNA was amplified using the following primers RPLP0 F-GTGTTCGACAATGGCAGCAT; RPLP0 R—GACACCCTCCAGGAAGCGA; SARS-CoV-2 Spike F-CCTACTAAATTAAATGATCTCTGCTTTACT; SARS-CoV-2 Spike R-CAAGCTATAACGCAGCCTGTA. Relative expression of SARS-CoV-2 Spike RNA was calculated by delta-delta-Ct by first normalizing to the housekeeping gene RPLP0 and then comparing to SARS-CoV-2 infected Vero E6 cells that were untreated (reference control). Curves were fit and inhibitory concentration (IC) IC50 and IC90 values calculated using Prism 8.

Immunofluorescence imaging and analysis. Formaldehyde fixed cells were washed with PBS and permeabilized for immunofluorescence using BD Cytofix/Cytoperm according to the manufacturers protocol for fixed cells and stained for SARS-CoV-2 with a primary anti-Nucleocapsid antibody (GeneTex GTX135357) followed by a secondary Goat anti Rabbit AF594 antibody (ThermoFisher A-11037) and nuclei stained with Sytox Green. Five or eight images per well were obtained using an Incucyte 53 (Sartorius) or Nikon Ti2-E microscope equipped with a Qi-2 camera and Lumencor Spectra III light engine respectively. The percent infected cells were calculated using built-in image analysis tools for the Incucyte S3. For images acquired with the Nikon Ti2, images were analysed using the Fiji distribution of ImageJ (PMID 22743772) and the DeepLearning plugin StarDist (Schmidt et al, 2012—see below) as follows. Channels were separated and the Sytox Green-stained nuclei were segmented using StarDist to generate individual masks for each nucleus. The AF594 channel (Nucleocapsid) was selected by removing background with a rolling ball of 50 pixels. A median filter (sigma=10) was applied to facilitate easier thresholding of AF594 signal. The resulting image was thresholded and a mask was generated representing all positive staining. Positive nuclei were selected by firstly eroding the StarDist generated nuclei mask by 2 pixels to reduce potential overlap with non-positive stain. Binary reconstruction was then carried out between the resulting mask and the AF594 mask. Uwe Schmidt, Martin Weigert, Coleman Broaddus, and Gene Myers. Cell Detection with Star-convex Polygons. International Conference on Medical Image Computing and Computer-Assisted Intervention (MICCAI), Granada, Spain, September 2018.

Proline distribution analysis. Protein amino acid sequences were downloaded from UniProtKB¹³. Proline distribution was analyzed using custom code in R (v3.6.0)¹⁴, including commands from the packages dplyr¹⁵ tidyr¹⁶, and stringr¹⁷. All plots were generated in ggplot2¹⁸. For individual protein plots, histograms were constructed with geom_histogram(binwidth=1) and kernel density estimations (KDEs) were constructed with geom_density(kernel=“gaussian”) and added as custom annotations (ggpubr package¹⁹). The bandwidth of KDEs for individual plots was assessed separately for each protein distribution using maximum likelihood cross-validation^{20,21} with the h.mlcv command from the kedd package²². The proline distribution score was calculated using the following formula:

Proline dist.score=log₁₀(B_{P}/B_{T}*P)

where B_{P} is the number of 10-amino acid blocks in a protein that contain one or more prolines (a protein with length 100 amino acids has 10 blocks); B_{T} is the total number of 10-amino acid blocks in a protein; and P is the total number of prolines in a protein. B_{P} values were obtained using custom code in R.

Data and Code availability. Data are deposited in the National Centre for Biotechnology Information GEO database under the accession number (GSE157036). Depicted structures have been solved and deposited: halofuginone PDB ID 4K88, ProSA PDB ID 5V58, and NCP22 PDB ID 5VAD. The custom-scripted macro used for automated image analysis of live cell imaging data is available from https://doi.org/10.26180/5f508284eb365. Code and files used for proline analysis can be found on https://github.com/jkccoker/Proline_analysis.

### Example 1: In Vivo Treatment of SARS-CoV-2 Infection

The purpose of this example is to demonstrate the in vivo efficacy of halofuginone against SARS-CoV-2 in a mammal.

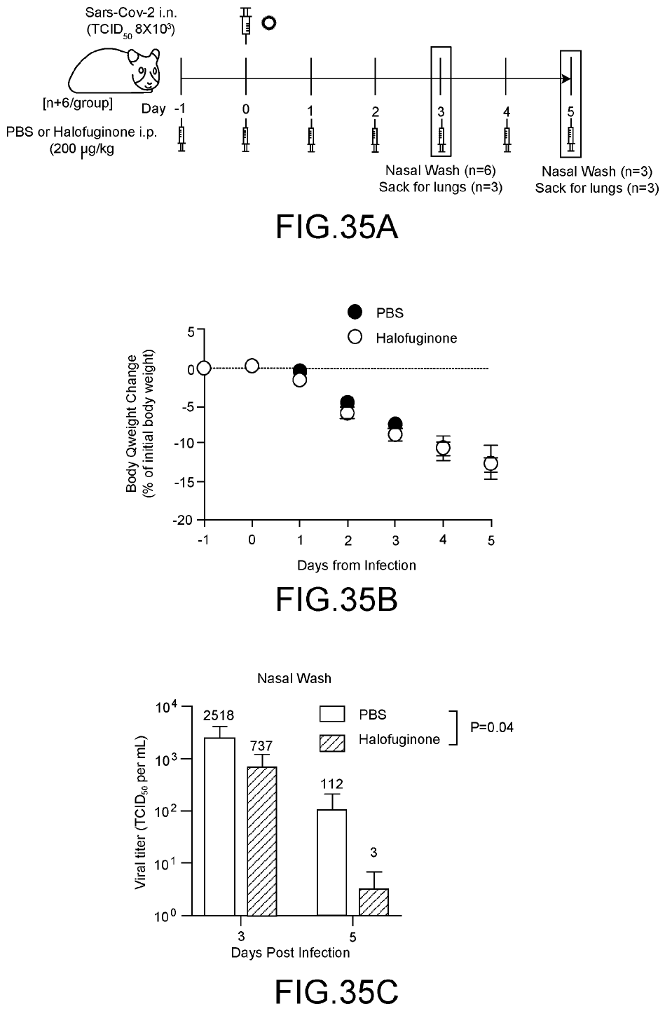

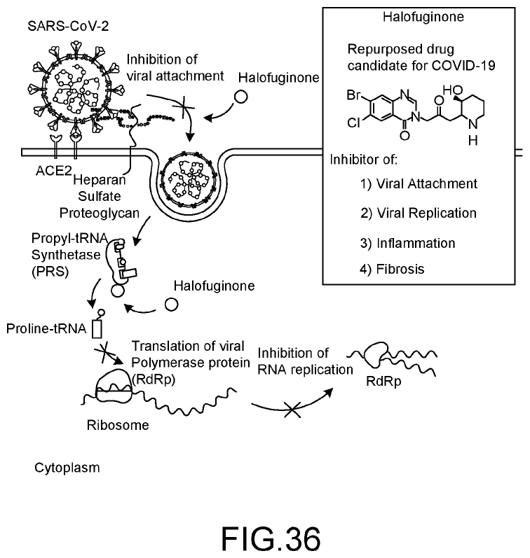

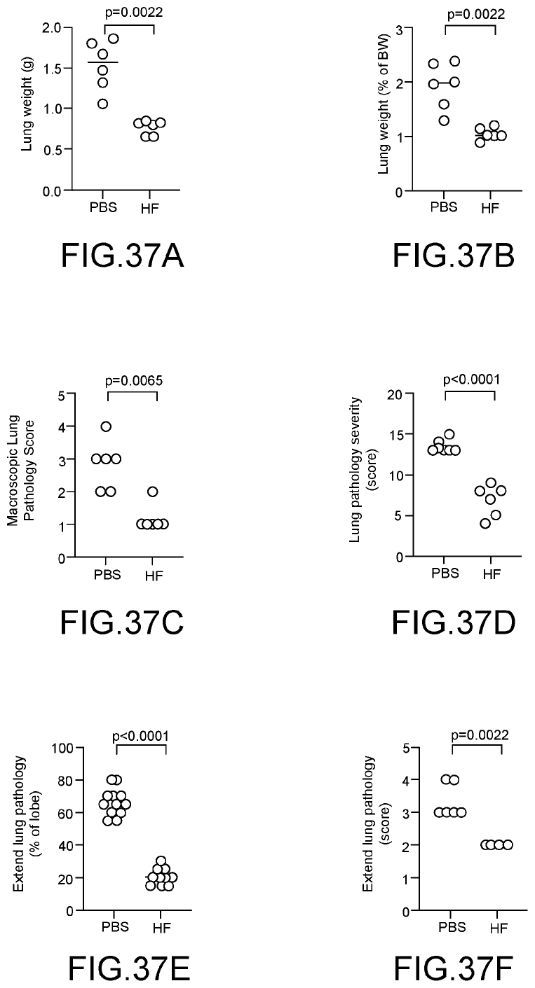

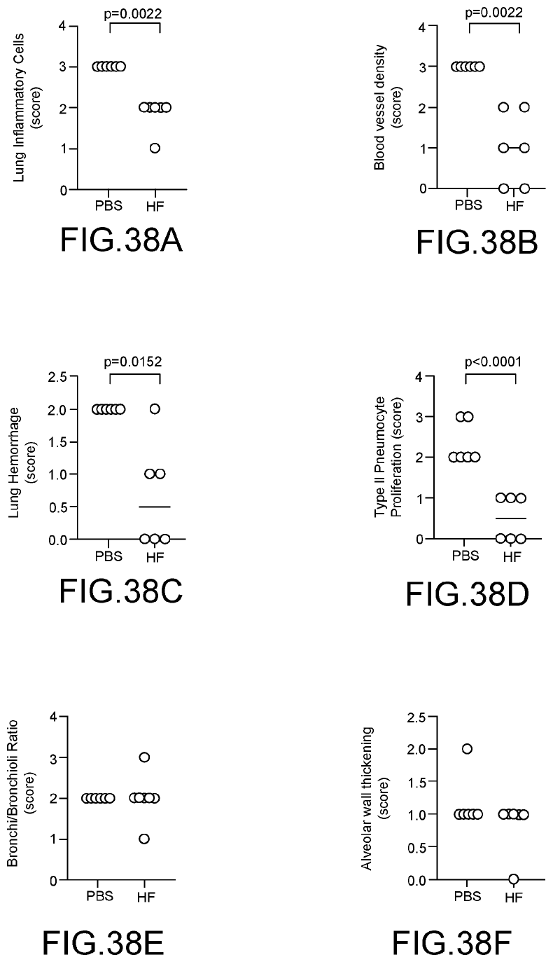

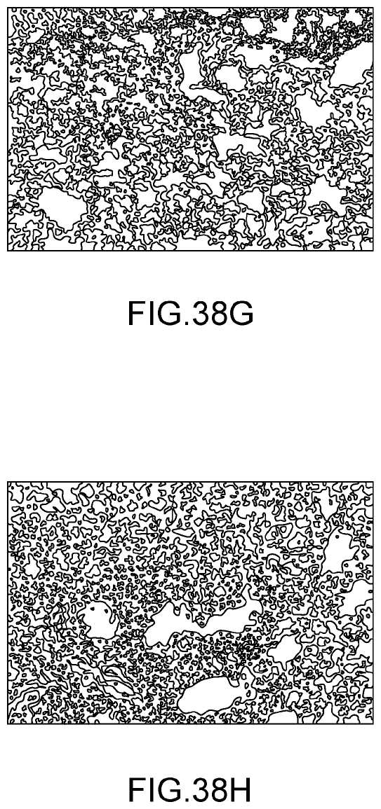

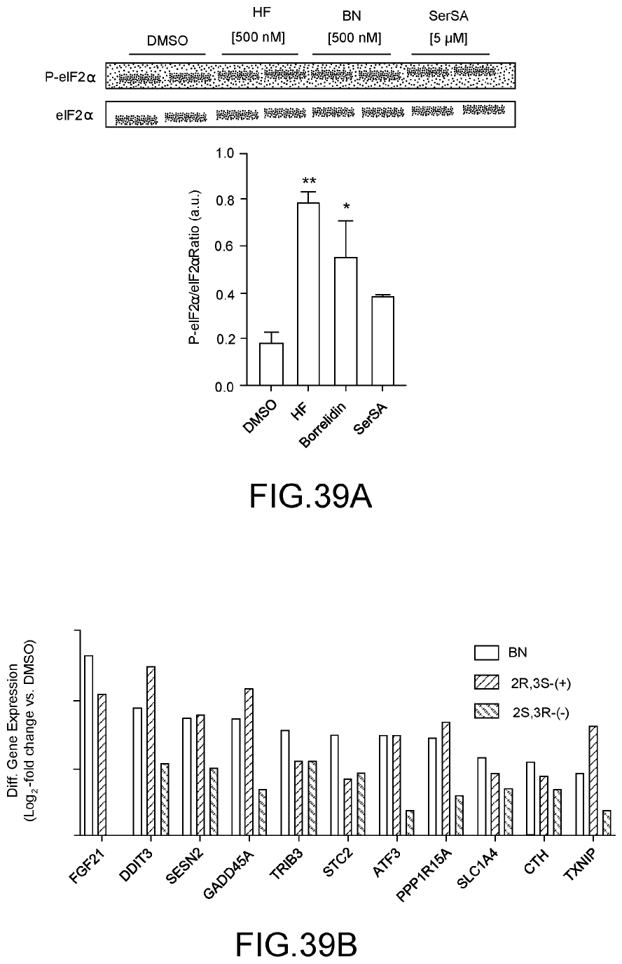

Six golden hamsters were dosed at 200 μg/kg with halofuginone starting at day −1, infected at day 0 with authentic SARS-COV-2 virus, and every day for five more days they received 200 μg/kg of halofuginone (FIG. 35A). Dosing was done intraperitoneally. A control group (n=6) was administered PBS. On day 3, half of the animals (n=3) in each group was sacrificed, and at day 5 the remaining animals (n=3) were sacrificed. TCID50 values as a measure of SARS-CoV-2 infectivity were measured from nasal washes at day 3 and day 5; the values were determined by viral cytopathic effect. Halofuginone exerted potent anti-viral efficacy in the infected animals (FIG. 35C).

### Example 2: Antiviral Activity of Halofuginone

The purpose of this example is to demonstrate antiviral activity of halofuginone in comparison to halofuginol and the following Boc-protected synthetic precursor used as the control compound in this example:

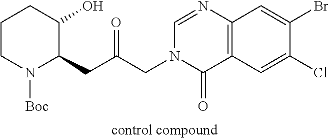

Experimental Procedure. The antiviral activity of 8 dilutions of each compound was explored by adding the drug to the assay cells 2 h after infection with the different viruses. Virus and compounds were left on the cells for the entire duration of the experiment (28 h for Zika virus and 24 h for all other viruses). The cytotoxicity of the same range of concentrations of compounds was determined by LDH release assay in the same cell lines.

Cell plating. Cells were detached and counted following SOP-RA 003 and SOP-RA 004. Count was recorded in the Cell Count Logbook, Volume 1. For each virus four plates were seeded, two for the antiviral assay and two for the cytotoxicity assay. Cells were seeded in 100 μl/well of complete media at the following densities:
Huh7: 8,000 cells/well (Dengue)
SH-SY5Y: 10,000 cells/wells (Zika)
HeLa Tzmbl: 8,000 cells/well (HIV)

After seeding, the plates were incubated at RT for 5 minutes for even distribution, and then at 37° C., 5% CO₂ until the following day.

Compounds dilutions. The 22.1 mM stocks were further diluted to 1 mM in DMSO by adding 211 μl of DMSO to 10 μl of compound. Three serial dilutions were prepared in different media for different viruses/cells. For each dilution, drugs were prepared at twice the final concentration in 50 μl volume, because they are diluted to the final concentration by an equal volume of virus or media.

Infection. Cells were infected in 50 μl of supplemented media/well at the following multiplicity of infection (MOI):
DENV-2, MOI 1
ZIKV, MOI 5
HIV, MOI 0.5

After removing culture media from cells, 50 μl of virus dilution were added to all wells except Column 11 (uninfected control), where the same volume of supplemented media was added. Cells were incubated for 2 h at 37C, 5% CO2. 50 μl of control compounds were added at the same time as the virus.

Compounds treatment. At the end of the 2 h incubation, 50 μl of diluted drugs were transferred from the dilution plate onto the cells. Virus and drugs were left on the cells for the entire duration of the experiment (24 h-28 h).

Fixation and development. After 28 h (ZIKV) or 24 h (all other viruses), the infection plates were washed with PBS, fixed for 30 mins with 4% formaldehyde, washed again with PBS, and stored in PBS at 4° C. until staining.

Infectivity readout. Cells were immunostained following SOP-RA 005. Briefly, any residual formaldehyde was quenched with 50 mM ammonium chloride, after which cells were permeabilised (0.1% Triton X100) and stained with an antibody recognising dengue virus E protein (Thermo Fisher Scientific MA1-27093), Zika virus E protein (Millipore MAB10216), or HIV-1 Gag protein (NIBSC ARP432). The primary antibodies were detected with an Alexa-488 conjugate secondary antibody (Life Technologies, A11001), and nuclei were stained with Hoechst. Images were acquired on a CellInsight CX5 high content platform (Thermo Scientific) using a 4× objective, and percentage infection calculated using CellInsight CX5 software (infected cells/total cells×100).

Determination of % Inhibition—IF assay. Normalized percentages of inhibition were calculated using the following formula:

$$100 \times$$

$$[ 1 - ( \frac{\% Infection Sample - \% Infection Uninfected Control}{\% Infection Infected Control - \% Infection Uninfected Control} ) ]$$

For control drugs, EC₅₀ values were extrapolated from the curves representing the best fit (non-linear regression analysis, variable slope) of the logarithm of compound concentration vs. the normalized percentages of inhibition, using GraphPad Prism (version 9).

Results. Halofuginone displayed antiviral activity against all viruses tested, with EC₅₀ ranging from 35.4 to 21.8 for DENV-2 (Dengue) and ZIKV, respectively, and 23.6 for HIV-1. An inactive intermediate in the synthesis of halofuginone did not display antiviral activity against any of the viruses tested. Halofuginol displayed antiviral activity only against HIV-1 (EC50 344.1 nM). Partial inhibition of DENV-2 (˜60%) was observed only at the highest concentration tested (1000 nM). No significant cytotoxicity was observed.

Numbered references cited in the Examples above:
1 Blight, K. J., McKeating, J. A. & Rice, C. M. Highly permissive cell lines for subgenomic and genomic hepatitis C virus RNA replication. J Virol 76, 13001-13014, doi:10.1128/jvi.76.24.13001-13014 2002 (2002).
2 Anower, E. K. F., Singh, G., Deng, Y., Gordts, P. & Esko, J. D. Corrigendum to: Triglyceride-rich lipoprotein binding and uptake by heparan sulfate proteoglycan receptors in a CRISPR/Cas9 library of Hep3B mutants. Glycobiology 30, 760, doi:10.1093/glycob/cwaa016 (2020).
3 Esko, J. D. in Current protocols in molecular biology (eds F. Ausubel et al.) 17.12.11-17.12.19 (Greene Publishing and Wiley-interscience, 1993).
4 Cribbs A P, L.-V. S., George C et al. CGAT-core: a python framework for building scalable, reproducible computational biology workflows. F1000Research 8:377 (2019).
5 Sims, D. et al. CGAT: computational genomics analysis toolkit. Bioinformatics 30, 1290-1291, doi:10.1093/bioinformatics/btt756 (2014).
6 Kim, D., Paggi, J. M. Park, C., Bennett, C. & Salzberg, S. L. Graph-based genome alignment and genotyping with HISAT2 and HISAT-genotype. Nat Biotechnol 37, 907-915, doi:10.1038/s41587-019-0201-4 (2019).
7 Liao, Y., Smyth, G. K. & Shi, W. The R package Rsubread is easier, faster, cheaper and better for alignment and quantification of RNA sequencing reads. Nucleic Acids Res 47, e47, doi:10.1093/nar/gkz114 (2019).
8 Heinz, S. et al. Simple combinations of lineage-determining transcription factors prime cis-regulatory elements required for macrophage and B cell identities. Mol Cell 38, 576-589, doi:10.1016/j.molcel.2010.05.004 (2010).
9 Gordts, P. et al. Reducing macrophage proteoglycan sulfation increases atherosclerosis and obesity through enhanced type I interferon signaling. Cell Metab 20, 813-826, doi:10.1016/j.cmet.2014.09.016 (2014).
10 Whitt, M. A. Generation of VSV pseudotypes using recombinant DeltaG-VSV for studies on virus entry, identification of entry inhibitors, and immune responses to vaccines. J Virol Methods 169, 365-374, doi:10.1016/j.jviromet.2010.08.006 (2010).
11 Jose, J., Taylor, A. B. & Kuhn, R. J. Spatial and Temporal Analysis of Alphavirus Replication and Assembly in Mammalian and Mosquito Cells. mBio 8, doi:10.1128/mBio.02294-16 (2017).
12 Schwarz, M. C. et al. Rescue of the 1947 Zika Virus Prototype Strain with a Cytomegalovirus Promoter-Driven cDNA Clone. mSphere 1, doi:10.1128/mSphere.00246-16 (2016).
13 UniProt, C. UniProt: a worldwide hub of protein knowledge. Nucleic Acids Res 47, D506-D515, doi:10.1093/nar/gky1049 (2019).
14 Team, R. C. R: A language and environment for statistical computing. R Foundation for Statistical Computing, Vienna, Austria. (2019).
15 H. Wickham, R. F., L. Henry, K. Müller. dplyr: A Grammar of Data Manipulation. R package version 0.8. https://CRAN.R-project.org/package=dplyr (2020).
16 Wickham, H. tidyr: Tidy Messy Data. R package version 1.1.2. https://CRAN.R-project.org/package=tidyr (2020).
17 Wickham, H. stringr: Simple, Consistent Wrappers for Common String Operations. R package version 1.4.0. https://CRAN.R-project.org/package=stringr (2019).
18 Wickham, H. ggplot2: Elegant Graphics for Data Analysis. Springer-Verlag New York. ISBN 978-3-319-24277-4, https://ggplot2.tidyverse.org (2016).
19 Kassambara, A. ggpubr: ‘ggplot2’ Based Publication Ready Plots. Package version 0.4.0. https://CRAN.R-project.org/package=ggpubr (2020).
20 J. D. F. Habbema, J. H., and K. Van den Brock. A stepwise discrimination analysis program using density estimation. Compstat 1974: Proceedings in Computational Statistics, Physica Verlag, Vienna (1974).
21 Duin, R. P. W. On the choice of smoothing parameters of Parzen estimators of probability density functions. IEEE Transactions on Computers C-25, 1175-1179 (1976).
22 Guidoum, A. C. kedd: Kernel estimator and bandwidth selection for density and its derivatives. R. Package version 1.0.3. http://CRAN.R-project.org/package=kedd (2015).

1. A method of treating a subject suffering from a disease or condition selected from the group consisting of a virus, a coronavirus, an iron-loading disease, an iron-deficiency disease, a lysosomal storage disease, a neurodegenerative disorder, a cancer, diabetes, or need for wound healing, comprising administering to the subject a therapeutically effective amount of halofuginone or a pharmaceutically acceptable salt thereof.

2. The method according to claim 1, wherein the disease or condition is a coronavirus.

3. The method according to claim 1, wherein the disease or condition is COVID-19.

4. The method according to claim 1, wherein the disease or condition is anemia, an iron-deficiency disease, or an iron-loading disease.

5. The method according to claim 1, wherein the disease or condition is a lysosomal storage disease.

6. The method according to claim 1, wherein the disease or condition is a neurodegenerative disease.

7. The method according to claim 6, wherein the neurodegenerative disease is Alzheimer's Disease.

8. A method of treating COVID-19 in a subject in need thereof comprising administering to the subject a therapeutically effective amount of halofuginone or a pharmaceutically acceptable salt thereof.

9. A method of treating COVID-19 in a subject in need thereof comprising administering to the subject a therapeutically effective amount of 2R,3S-(+) halofuginone or a pharmaceutically acceptable salt thereof.

10. A method of treating COVID-19 in a subject in need thereof comprising administering to the subject a therapeutically effective amount of 2R,3S-(+) halofuginone or a pharmaceutically acceptable salt thereof, or a pharmaceutical composition comprising a therapeutically effective amount of 2R,3S-(+) halofuginone or a pharmaceutically acceptable salt thereof.

11. The method according to claim 9, wherein the therapeutically effective amount of 2R,3S-(+) halofuginone or a pharmaceutically acceptable salt thereof is a dose of about 1 μg/kg to about 20 μg/kg.

12. The method according to claim 9, wherein the dose is about 1 μg/kg, about 1.5 μg/kg, about 2 μg/kg, about 2.5 μg/kg, about 3 μg/kg, about 4 μg/kg, about 5 μg/kg, about 10, or about 15 μg/kg.

13. The method according to claim 9, wherein the therapeutically effective amount is about 0.01 mg to about 0.5 mg.

14. The method according to claim 9, wherein the therapeutically effective amount is about 0.050 mg, about 0.100 mg, about 0.125 mg, about 0.150 mg, about 0.175 mg, about 0.200 mg, about 0.225 mg, about 0.250 mg, about 0.275 mg, about 0.300 mg, about 0.325 mg, about 0.350 mg, about 0.375 mg, or about 0.400 mg.

15. The method according to claim 1, wherein the therapeutically effective amount leads to the inhibition of spike protein binding.

16. The method according to claim 15, wherein the spike protein binding is inhibited by about 10% to about 90%.

17. The method according to claim 1, wherein the therapeutically effective amount leads to a reduction of cell surface heparan sulfate content.

18. The method according to according to claim 1, wherein cell surface heparan sulfate content is reduced by about 2-fold to about 10-fold.

19. The method of claim 1, wherein the subject is human.
